(19) United States
(12) Patent Application Publication
(10) Pub. No.: US 2008/0091035 A1
(43) Pub. Date: Apr. 17, 2008

(54) Process for the preparation of a borylated thiophene

(75) Inventors: Milton R. Smith III, East Lansing, MI (US); Robert E. Maleczka JR., DeWitt, MI (US); Ghayoor Chotana, East Lansing, MI (US); Venkata Kallepalli, East Lansing, MI (US); Sulagna Paul, Danbury, CT (US)

Correspondence Address: Ian C. McLeod;IAN C. McLEOD, P.C., 2190 Commons Parkway, Okemos, MI, 48864, US

(73) Assignee: Board of Trustees of Michigan State University, Lansing, MI (US)

(21) Appl. No.: 11/900,330

(22) Filed: Sep. 11, 2007

Related U.S. Application Data
(60) Provisional application No. 60/843,589, filed on Sep. 11, 2006.

(51) Int. Cl. C07D 333/30

(52) U.S. Cl. 549/4

(57) ABSTRACT

Borylated thiophenes are prepared by the process. The borylated thiophenes are intermediates to thiophene polymers for electronic applications.

### CROSS-REFERENCE TO RELATED APPLICATIONS

This application claims benefit to U.S. Provisional Application Ser. No. 60/843,589, filed Sep. 11, 2006, which is incorporated herein by reference in its entirety.

### STATEMENT REGARDING FEDERALLY SPONSORED RESEARCH OR DEVELOPMENT

This work was supported by a grant from the National Institute of Health (NIH)—Grant No. GM063188. The U.S. Government has certain rights to this invention.

### BACKGROUND OF THE INVENTION

(1) Field of the Invention

The present invention relates to the preparation of borylated thiophenes which are intermediates to thiophene polymers for electronic applications. Novel substituted thiophenes are described.

(2) Description of the Related Art

Thiophenes have important applications ranging from advanced materials, such as light emitting diodes^{a} and sensors,^{b} to medical applications as therapeutic agents.^{c,d} Consequently, synthesis of synthetic intermediates is an area of intense interest.

One approach to preparing substrates for advanced applications is the Ir-catalyzed borylation of thiophene C—H bonds, which has been demonstrated for a limited of compounds.^{e,f} What limits applications of this method to synthesis of advanced materials^{g} and pharmaceutical agents, is the limited reaction scope for thiophenes, where substitutions that are compatible for arene and N- and O-containing heterocycles can completely deactivate the catalyst.^{h} In addition, the borylation of 3-substituted thiophenes offer little regiochemical control in many instances.

### OBJECTS

It is an object of the present invention to provide borylated thiophenes as intermediates to thiophene polymers. It is further an object of the present invention to provide a process for the preparation of the thiophenes which is novel, on high yield.

These and other objects will become increasingly apparent by reference to the following description and drawings.

### SUMMARY OF THE INVENTION

The present invention provides a process for producing a boryl-mono-, di- or tri-substituted thiophene (I), which comprises: reacting a mono-, di- or tri-substituted thiophene (II) in a reaction mixture with a non-reactive solvent selected from, but not limited to, aliphatic hydrocarbons and ethers at elevated temperatures between about 0 and 150° C. with an HB or B—B organic compound, in the presence of a catalytically effective amount of an iridium complex catalytic composition comprising an iridium complex of the formula: (BY)ₙ—Ir-(ligand)ₘ where n is equal to one to five and m is equal to one to three, excluding hydrogen, bonded to the iridium BY is a boron moiety and the ligand is selected from the group consisting of a phosphorus organic ligand, an organic amine, an imine, a nitrogen heterocycle, and an ether wherein the ligand is at least in part bonded to the iridium, to form the mono-, di- or tri-substituted thiophene (I) in the reaction mixture; and evaporating the solvent and portions of the reaction mixture which are volatile from the reaction mixture to produce the boryl-mono-, di- or tri-substituted thiophene (I).

The present invention provides a process for producing a 2-boryl-5-substituted thiophene (I), which comprises: reacting a thiophene (II) with an HB or B—B organic compound in a reaction mixture with a non-reactive first solvent which is a non-solvent for the 2-boryl-5-substituted thiophene (I) at elevated temperatures between about 0 and 150° C. in the presence of a catalytically effective amount of an iridium complex catalytic composition comprising an iridium complex of the formula: (BY)ₙ—Ir-(ligand)ₘ where n is equal to one to five and m is equal to one to three, excluding hydrogen, bonded to the iridium BY is a boron moiety and the ligand is selected from the group consisting of a phosphorus organic ligand, an organic amine, an imine, a nitrogen heterocycle, and an ether wherein the ligand is at least in part bonded to the iridium, in a molar ratio of complex to ligand between 1 to 3 and 1 to 1, wherein the ligand is at least in part bonded to the iridium, to form the 2-boryl-5-substituted thiophene (I); evaporating the first solvent and portions of the reaction mixture which are volatile from the reaction mixture; dissolving the 2-boryl-5-substituted thiophene in a second solvent; and isolating the 2-boryl-5-substituted thiophene (I) from the second solvent.

The present invention provides a 2-boryl-5-substituted thiophene (I) wherein there is at least one ring substituent in the 5 position other than hydrogen selected from the group consisting of boryl, halo other than fluoro, cyano, alkyl, alkoxy, thioalkyl, amino alkyl, acyl, alkyl aminoacyl, trifluoro alkyl, aryl, alkyl silyl, and containing 1 to 8 carbon atoms except for the halo group and boryl group and wherein the boryl group is derived from HBPin or B₂Pin.

The present invention provides a process for producing 2-boryl-5-substituted thiophene (I), which comprises: reacting a 2-boryl-5-substituted thiophene (II) with HBPin or B₂Pin₂, in a reaction mixture with a non-reactive first solvent which is a non-solvent for the 2-boryl-5-substituted thiophene (I) at elevated temperatures between about 0 and 150° C. in the presence of a catalytically effective amount of an iridium complex catalytic composition comprising an iridium complex of the formula: (BY)ₙ—Ir-(ligand)ₘ where n is equal to one to five and m is equal to one to three, excluding hydrogen, bonded to the iridium BY is a boron moiety and the ligand is selected from the group consisting of a phosphorus organic ligand, an organic amine, an imine, a nitrogen heterocycle, and an ether wherein the ligand is at least in part bonded to the iridium, in a molar ratio of complex to ligand between 1 to 3 and 1 to 1, and wherein the ligand is at least in part bonded to the iridium, to form the 2-boryl-5-substituted thiophene (I) in the reaction mixture; and evaporating the solvent and portions of the reaction mixture which are volatile from the reaction mixture to produce the 2-boryl-5-substituted thiophene (I). In further embodiments, there is at least one ring substituent for 5-substituted other than hydrogen selected from the group consisting of boryl, halo other than fluoro, cyano, alkyl, alkoxy, thioalkyl, amino alkyl, acyl, alkyl aminoacyl, trifluoro alkyl, aryl, alkyl silyl, and containing 1 to 8 carbon atoms except for the halo group and boryl group and the boryl group is derived from HBPin or B₂Pin. In further still embodiments, the ligand is selected from the group consisting of:

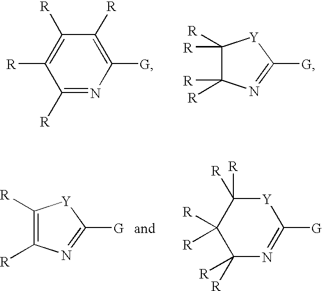

wherein R are each selected from the group consisting of hydrogen, linear alkyl containing 1 to 8 carbon atoms, branched alkyl containing 1 to 8 carbons, and a carbon in a cyclic structure, Y is a carbon, oxygen, nitrogen, or sulfur containing moiety, and G is a heteroatom containing group, multiple atom chain, or multiple atom ring. In still further embodiments, the ligand is selected from the group consisting of:

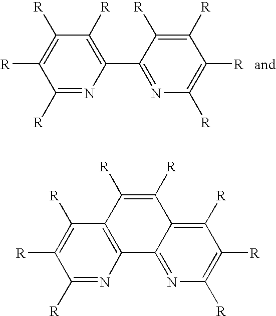

wherein R are each selected from the group consisting of hydrogen, linear alkyl containing 1 to 8 carbon atoms, branched alkyl containing 1 to 8 carbons, and a carbon in a cyclic structure. In still further embodiments, the ligand is selected from the group consisting of:

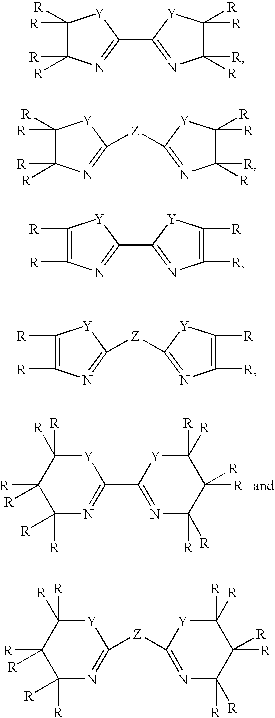

wherein R are each selected from the group consisting of hydrogen, linear alkyl containing 1 to 8 carbon atoms, branched alkyl containing 1 to 8 carbons, and a carbon in a cyclic structure, Y is a carbon, oxygen, nitrogen, or sulfur containing moiety, and Z is a carbon, oxygen, nitrogen, sulfur, or boron containing moiety or a multiple atom chain containing a carbon, oxygen, nitrogen, sulfur, or boron containing moiety. In further still embodiments, the HB or B—B organic compound is HBPin or B₂Pin₂. In further still embodiments, the complex is an iridium complex of [Ir(OMe)(COD)]₂, where COD is 1,5-cyclooctadine complexed with 4,4-di-t-butyl-2,2′bipyridine (d^{t}bpy). In still further embodiments, the ligand is bisoxazoline.

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

2-substituted thiophenes were borylated using 3 mol % [Ir] catalysts loading at room temperature. Typically, borylation was complete within 1 h and 5-borylated products were isolated in 82-97% yields (Table 1). Apart from common functionalities in iridium catalyzed borylation such as C₁, CO₂Me, I, OMe, additional functional group tolerance to ketone (COMe), and trimethylsilyl (TMS) groups was also observed. 2,3-di-halo substituted thiophene (Table 1, entry 7) was also cleanly borylated under these conditions.

TABLE 1
Catalytic Borylation of 2-Substituted Thiophenes.

| Entry | Thiophene | Product | % Yield |
|---|---|---|---|
| 1 | 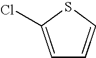 | 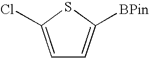 | 97 |
| 2 | 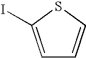 | 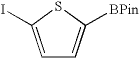 | 92 |
| 3 | 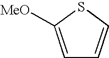 | 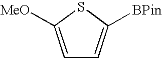 | 82 |
| 4 | 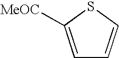 | 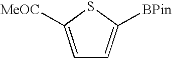 | 85 |
| 5 | 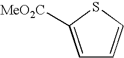 | 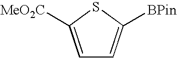 | 94 |
| 6 | 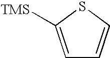 | 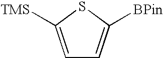 | 93 |
| 7 | 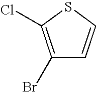 | 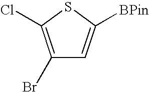 | 78 |

The case becomes slightly complicated for 3-substituted thiophenes since there are two open positions adjacent to the heteroatom, which can potentially be borylated. The observed regioselectivities are shown in Table 2. For smaller substituents such as Cl, Br, and Me, mixtures of two borylated regioisomers were observed. The major isomer observed for these substituents was on the 5-position. The presence of small amounts of minor borylated isomer at the 2-position indicates that the steric effects of ortho substituents decrease for 5-membered rings relative to 6-membered rings.

In the case of 3-cyanothiophene, the ratio of 2 to 5-boryalted isomers was 53:47 respectively. This was surprising as we expected 5-borylated isomer to be the major product. One possible explanation could be that the electron withdrawing inductive effect of the cyano group at the 2-position makes it much more activated as compared to the 5-position. Small steric demand of cyano group and wider bond angles in 5-membered ring may also facilitate borylation at the 2-position. Claus Christophersen⁴ has reported the preparation of the major isomer here by Pd catalyzed borylation of 2-bromo-3-cyanothiophene. In their case, although the borylated product was formed in 45% yield based on NMR, all attempts to isolate the product failed due to complete deborylation during aqueous workup. No such deborylation was observed in our case and the product mixture was isolated in 54% yield.

For sterically bulky substituents, such as ketone, ester, and TMS, a single monoborylated isomer was observed. Phenyl group also gives 97% regioselectivity for the 5-borylated isomer. Good to excellent regioselectivities for the 5-position in 3-substituted thiophenes observed here are consistent with sterically directed aromatic borylation and silylation.^{2,3}

TABLE 2
Catalytic Borylation of 3-Substituted Thiophenes.

| | | Products | | Ratio | |
|---|---|---|---|---|---|
| Entry | Thiophene | A | B | A:B | % Yield |
| 1 | 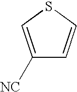 | 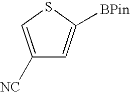 | 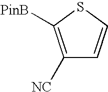 | 47:53 | 54 |
| 2 | 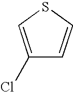 | 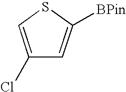 | 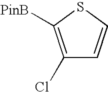 | 78:22 | 66 |
| 3 | 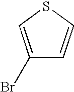 | 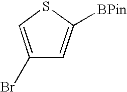 | 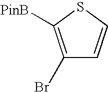 | 93:7 | 63 |
| 4 | 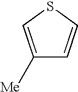 | 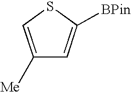 | 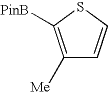 | 89:11 | 68 |
| 5 | 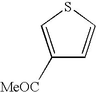 | 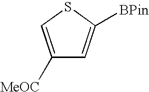 | — | >99:1 | 82 |
| 6 | 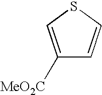 | 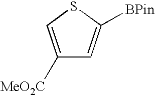 | — | >99:1 | 95 |
| 7 | 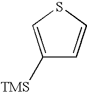 | 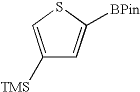 | — | >99:1 | 79 |
| 8 | 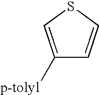 | 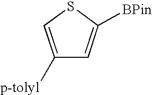 | 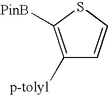 | 97:3 | 74 |

Next we examined the borylation of 2,5-di-substituted thiophenes. There is only one borylated product regioisomer possible for symmetrical substrates. We noted some complications for electron deficient as well as electron rich thiophenes due to different reasons. Borylation of 2,5-di-chlorothiophene became sluggish after initial rapid conversion, with observation of brown particles indicating the decomposition of catalyst. Nevertheless the conversion was complete in 20 h and the product was isolated in 86% yield. The borylation of 2,5-di-bromothiophene was more problematic, and only 89% conversion of the substrate was observed after 48 h at room temperature with 9 mol % [Ir] catalyst loadings. The monoborylated product was isolated in 56% yield. The reason for reduced catalytic activity after rapid initial conversion could be that the C-halogen bonds in these cases are weak, and may compete with the desired C—H activation. Attempted catalytic borylation of electron rich 2,5-di-methylthiophene using [Ir(OMe)(COD)]/d^{t}bpy system at room temperature was also very slow. Reduced activity due to electron rich behavior in this case was over come by using (Ind)Ir(COD)/dmpe system at 150° C. and the monoborylated product was isolated in 97% yield.

Unsymmetrical 2,5-disubstituted thiophenes yielded regioisomeric mixtures of two monoborylated products (Table 3). The borylation takes place preferentially ortho to the less bulky substituents. When the steric demands of the two substituents are sufficiently different, as in the case of 2,chloro-5-trimethylsilylthiophene, a single monoborylated product was isolated in 93% yield. Attempted borylations of 2-chloro-5-acetyl thiophene and 2-bromo-5-acetyl thiophene were unsuccessful and the reactions usually stopped after ˜10% conversion.

TABLE 3
Catalytic Borylation of 2,5-di-Substituted Thiophenes.

| | | Products | | Ratio | |
|---|---|---|---|---|---|
| Entry | Thiophene | A | B | A:B | % Yield |
| 1 | 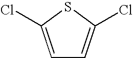 | 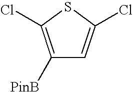 | — | — | 82 |
| 2 | 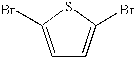 | 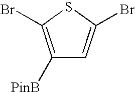 | — | — | 56 |
| 3 | 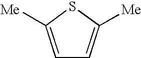 | 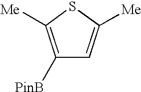 | — | — | 97 |
| 4 | 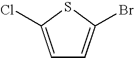 | 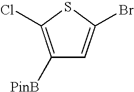 | 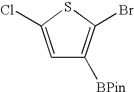 | 67:33 | 87 |
| 5 | 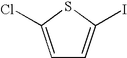 | 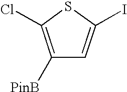 | 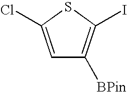 | 85:15 | 89 |
| 6 | 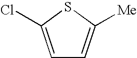 | 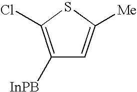 | 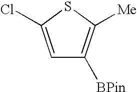 | 70:30 | 86 |
| 7 | 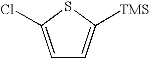 | 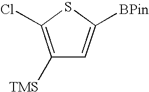 | — | >99:1 | 91 |

The high regioselectivity of borylation for 2,chloro-5-trimethylsilylthiophene prompted us to examine the diborylation of 2-substituted thiophenes. We reasoned that since the BPin group attached to the 5-position via monoborylation is significantly bulkier than the 2-substituent, the second borylation should regioselectively take place at the 3-position. Indeed, diborylation of 2-substituted thiophenes was found to be highly regioselective (Table 4).

In case of 2-methyl thiophene and 2-methoxy thiophene, small amounts (1.5-1.6% by GC-FID) of minor diborylated isomer were also observed. The GC-FID retention times of these minor diborylated isomers were different from those observed for diborylated products derived from 3-methyl/methoxy substituted thiophenes. Therefore the minor diborylated isomers observed for 2-methyl/methoxy substituted thiophenes could either be 4,5-diborylated or methyl/methoxy borylated products.

TABLE 4
Diborylation of 2-Substituted Thiophenes.

| Entry | Thiophene | Product | % Yield |
|---|---|---|---|
| 1 | 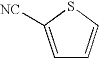 | 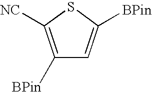 | 88 |
| 2 | 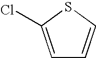 | 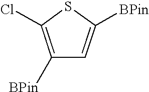 | 85 |
| 3 | 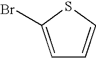 | 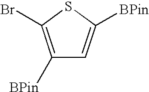 | 92 |
| 4 | 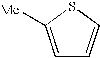 | 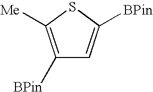 | 90 |
| 5 | 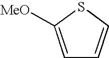 | 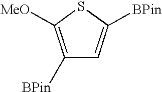 | 89 |

Attempted diborylation of 2-trimethylsilyl thiophene resulted in only 12% diborylation (by GC-FID) after 72 h at 60° C. Similarly, attempted diborylation of methyl-2-thiophene carboxylate resulted in only 7% diborylation (mixture of two isomers in 63:37 ratio by GC-FID) after 36 h at room temperature. These results suggest that diborylation is only feasible when the 2-substituent is relatively small.

3-substituted thiophenes can also be diborylated at 2 and 5-positions when the 3-substituent is CN, Cl, Br, Me, and p-tolyl (Table 5). 3-trimethylsilyl thiophene went to only 18% diborylation after 48 h at 60° C., while attempted borylation of 3-acetyl thiophene with 3 equivalents of HBPin resulted in reduction of the carbonyl group during diborylation.

It is worthwhile to note that during attempted diborylation of 2 and 3-trimethylsilyl thiophenes, formation of small amounts of B₂Pin₂ was observed from HBPin.

TABLE 5
Diborylation of 3-Substituted Thiophenes.

| Entry | Thiophene | Product | % Yield |
|---|---|---|---|
| 1 | 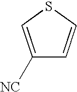 | 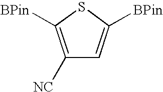 | 85 |
| 2 | 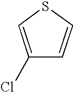 | 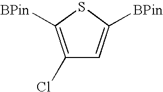 | 91 |
| 3 | 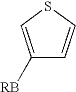 | 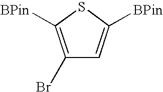 | 95 |
| 4 | 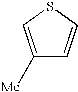 | 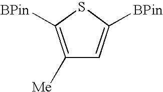 | 77 |
| 5 | 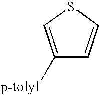 | 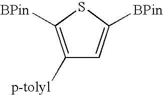 | 61 |

In case of 2,3,5-tri substituted thiophenes, the 4-position is locked between two ortho substituents. Since the bond angels in 5-membered heterocycles are wider than those in 6-membered rings, we thought that the 4-position in 2,3,5-tri substituted thiophenes might be accessible for borylation. However, only trace amount of borylation was observed for 3-bromo-2,5-di-methyl thiophene at room temperature conditions using [Ir(OMe)(COD)]₂ and d^{t}bpy. The outcome was same with (Ind)Ir(COD) and dmpe system at 150° C. Apart from steric hindrance for borylation, electron rich nature of 3-bromo-2,5-di-methyl thiophene could also be responsible for this low reactivity. Borylation of 3-bromo-2,5-di-chloro thiophene, an electron deficient substrate, was tested using (Ind)Ir(COD) and dmpe at 150° C. The result was surprising as the single product obtained in 73% isolated yield was found to be 3-bromo-2-chloro-5-(4,4,5,5-tetramethyl-1,3,2-dioxaboryl)-thiophene.

The product obtained here was found to be identical to one obtained by borylation of 3-bromo-2-chloro thiophene (Table 1, entry 7). The result was interesting for several reasons. Firstly, instead of C—H bond, a C-Halogen bond is activated by Iridium catalyst. Secondly, a C—Cl bond was activated in preference to C—Br bond. Finally, one of the two C—Cl bonds, which is sterically more accessible, was selectively activated.

There are at least two pathways for the observed product. The product can either be formed by oxidative addition of C—Cl bond followed by reductive elimination of C—B bond. Another possibility is first reduction of C—Cl bond to C—H bond followed by rapid borylation. The mechanism of this reaction needs to be fully investigated.

Since 2,3,5-trisubstituted thiophenes could not be borylated to synthesize tetra-substituted thiophenes, we looked for other possible routs for the final product. Borylation of 2,5-di substituted thiophene (Table 3) followed by bromination can also give the desired product. Although bromination of aryl boronic esters is unknown, there are few examples in literature where a aryl/heteroaryl boronic acid is brominated. R We were successful in brominating 2,5-dimethyl-3-(4,4,5,5-tetramethyl-1,3,2-dioxaboryl)-thiophene using one equivalent of Br₂ in CHCl₃ and the mono brominated product 3-bromo-2,5-dimethyl-4(4,4,5,5-tetramethyl-1,3,2-dioxaboryl)-thiophene was isolated in 82% yield (FIG. 2). Use of slight excess of bromine results in the bromination of methyl groups and hence should be avoided.

Bromination of 2,5-chloro-3-(4,4,5,5-tetramethyl-1,3,2-dioxaboryl)-thiophene was found not possible either with Br₂ in CHCl₃ or NBS in acetonitrile. During our search for different routs for the desired tetra-substituted thiophene product of this substrate, we found that trimethylsilyl group can easily be replaced with Br using NBS in acetonitrile (FIG. 3).

The intermediate thiophene boronic esters can be employed in subsequent Suzuki coupling without isolation.

Further studies regarding Suzuki coupling with hetero aryl halides, borylation of Suzuki coupled thiophenes, and electrophillic aromatic substitution (bromination, nitration etc) of aryl/heteroaryl boronic esters needs to be extensively explored.

All commercially available chemicals were used as received or purified as described. Bis(η⁴-1,5-cyclooctadiene)-di-μ-methoxy-diiridium(I) [Ir(OMe)(COD)]₂ and (η⁵-Indenyl)(cyclooctadiene)iridium {(Ind)Ir(COD)} were prepared per the literature procedures.^{i,j} Pinacolborane (HBPin) was generously supplied by BASF. 4,4′-Di-t-butyl-2,2′-bipyridine (d^{t}bpy) was purchased from Aldrich. All substrates were purified before use. Solid substrates were sublimed under vacuum. Liquid substrates were distilled before use. Pinacolborane (HBPin) was distilled before use. n-Hexane was refluxed over sodium, distilled, and degassed. Dimethoxy ethane (DME), ether, and tetrahydrofuran were obtained from dry stills packed with activated alumina and degassed before use. Silica gel was purchased from EMD™ (230-400 Mesh).

General Methods:

All reactions were monitored by GC-FID (Varian CP-3800; column type: WCOT Fused silica 30 m×0.25 mm ID coating CP-SIL 8 CB). GC-FID method: 70° C., 2 min.; 20° C./min, 9 min.; 250° C., 20 min.; All reported yields are for isolated materials.

¹H and ¹³C NMR spectra were recorded on a Varian Inova-300 (300.11 and 75.47 MHz respectively), Varian VXR-500 or Varian Unity-500-Plus spectrometer (499.74 and 125.67 MHz respectively) and referenced to residual solvent signals (7.24 ppm and 77.0 ppm for CDCl₃, respectively). ¹¹B spectra were recorded on a Varian VXR-300 operating at 96.29 MHz and were referenced to neat BF₃.Et₂O as the external standard. All coupling constants are apparent J values measured at the indicated field strengths. All 2-dimensional experiments were run using z-axis pulse field gradients. Elemental analyses were performed at Michigan State University using a Perkin Elmer Series II 2400 CHNS/O Analyzer. GC-MS data were obtained using a Varian Saturn 2200 GC/MS (column type: WCOT Fused silica 30 m×0.25 mm ID coating CP-SIL 8 CB). High-resolution mass spectra were obtained at the Mass Spectrometry Core of the Research Technology Support Facility (RTSF) at Michigan State University. Melting points were measured on a MEL-TEMP® capillary melting apparatus and are uncorrected. Regiochemistry of the borylated products was assigned by NMR spectroscopy (¹H, ¹³C, gHMQC, gHMBC).

Experimental Details and Spectroscopic Data:

2. Syntheses of Substrates:

### a. 2-Trimethylsilylthiophene

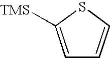

2-Trimethylsilylthiophene was prepared per the literature procedure.^{k} The product was isolated as colorless oil (31-33° C. at 0.01 mm Hg, 2.62 g, 56% yield). ¹H NMR (CDCl₃, 500 MHz): δ 7.58 (dd, J=4.6, 0.8 Hz, 1H), 7.25 (dd, J=3.3, 0.8 Hz, 1H), 7.17 (dd, J=4.6, 3.3, Hz, 1H), 0.31 (s, 9H, CH₃ of TMS); ¹³C NMR {¹H} (CDCl₃, 125 MHz): δ 140.1 (C), 133.9 (CH), 130.4 (CH), 128.1 (CH), −0.01 (3 CH₃ of TMS).

### b. 3-Trimethylsilylthiophene

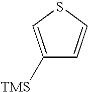

3-Trimethylsilylthiophene was prepared per the literature procedure.^{l} The product was isolated as colorless oil (34° C. at 0.01 mm Hg, 1.77 g, 57% yield). ¹H NMR (CDCl₃, 500 MHz): δ 7.42 (dd, J=2.6, 1.1 Hz, 1H), 7.38 (dd, J=4.8, 2.6 Hz, 1H), 7.17 (dd, J=4.8, 1.1 Hz, 1H), 0.25 (s, 9H, CH₃ of TMS); ¹³C NMR {¹H} (CDCl₃, 125 MHz): δ 141.2 (C), 131.39 (CH), 131.37 (CH), 125.6 (CH), −0.6 (3 CH₃ of TMS).

### c. 3-p-Tolylthiophene

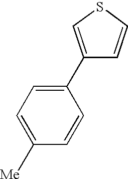

3-p-Tolylthiophene^{m} was prepared by the Suzuki coupling of 3-bromothiophene and p-tolylboronic acid. The product was isolated as a white solid (629 mg, 90% yield). ¹H NMR (CDCl₃, 500 MHz): δ 7.47-7.50 (m, 2H), 7.39-7.40 (m, 1H), 7.37-7.36 (m, 2H), 7.20-7.19 (m, 2H), 2.36 (s, 3H); ¹³C NMR {¹H} (CDCl₃, 125 MHz): δ 142.3 (C), 136.8 (C), 133.1 (C), 129.4 (CH), 126.3 (2 CH), 126.0 (CH), 119.6 (CH), 21.1 (CH₃).

### d. 2-Chloro-5-trimethylsilylthiophene

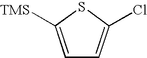

2-Chloro-5-trimethylsilylthiophene was prepared by following the literature procedure for the synthesis of 2-bromo-5-trimethylsilylthiophene.ⁿ The product was isolated as colorless oil (56-57° C. at 0.01 mm Hg, 2.61 g, 69% yield). ¹H NMR (CDCl₃, 500 MHz): δ 6.98 (d, J=3.5 Hz, 1H), 6.93 (d, J=3.5 Hz, 1H), 0.27 (s, 9H, CH₃ of TMS); ¹³C NMR {¹H} (CDCl₃, 125 MHz): δ 140.2 (C), 134.5 (C), 133.3 (CH), 127.4 (CH), −0.3 (3 CH₃ of TMS); FT-IR (neat) \tilde{ν}ₘₐₓ: 2959, 1415, 1251, 1205, 1072, 964, 841 cm⁻¹; GC-MS (EI) m/z (% relative intensity): M⁺ 190 (34), 192 (13), 175 (100); Anal. Calcd for C₇H₁₁ClSSi: C, 44.07; H, 5.81. Found: C, 43.59; H, 5.90; HRMS (EI): m/z 190.0036 [M⁺; Calcd for C₇H₁₁ClSSi: 190.0039]

3. Catalytic Borylation of Substituted Thiophenes Borylation Using d^{t}bpy Ligand

General Procedure A (Monoborylation with Heteroaromatic Substrate as the Limiting Reactant)

The Ir-catalyst was generated by a modified literature protocol,^{o} where in a glove box, two separate test tubes were charged with [Ir(OMe)(COD)]₂ (10 mg, 0.015 mmol, 3 mol % Ir) and d^{t}bpy (8 mg, 0.03 mmol, 3 mol %). Excess HBPin (1.5 to 2 equiv) was added to the [Ir(OMe)(COD)]₂ test tube. n-Hexane (1 mL) was added to the d^{t}bpy containing test tube in order to dissolve the d^{t}bpy. The d^{t}bpy solution was then mixed with the [Ir(OMe)(COD)]₂ and HBPin mixture. After mixing for one minute, the resulting solution was transferred to the 20 mL scintillation vial equipped with a magnetic stirring bar. Additional n-hexane (2×1 mL) was used to wash the test tubes and the washings were transferred to the scintillation vial. 2-Substituted heterocyclic substrate (1 mmol, 1 equiv) was added to the scintillation vial. The reaction was stirred at room temperature and was monitored by GC-FID/MS. After completion of the reaction, the volatile materials were removed on a rotary evaporator. The crude material was dissolved in CH₂Cl₂ and passed through a short plug of silica to afford the corresponding borylated product.

General Procedure B (Monoborylation with HBPin as the Limiting Reactant)

In a glove box, two separate test tubes were charged with [Ir(OMe)(COD)]₂ (10 mg, 0.015 mmol, 3 mol % Ir) and d^{t}bpy (8 mg, 0.03 mmol, 3 mol %). HBPin (1 mmol, 1 equiv) was added to the [Ir(OMe)(COD)]₂ test tube. n-Hexane (1 mL) was added to the d^{t}bpy containing test tube in order to dissolve the d^{t}bpy. The d^{t}bpy solution was then mixed with the [Ir(OMe)(COD)]₂ and HBPin mixture. After mixing for one minute, the resulting solution was transferred to the 20 mL scintillation vial equipped with a magnetic stirring bar. Additional n-hexane (2×1 mL) was used to wash the test tubes and the washings were transferred to the scintillation vial. Excess 2-substituted heterocyclic substrate (2-4 equiv) was added to the scintillation vial. The reaction was stirred at room temperature and was monitored by GC-FID/MS. After completion of the reaction, the volatile materials were removed on a rotary evaporator. The crude material was dissolved in CH₂Cl₂ and passed through a short plug of silica to afford the corresponding borylated isomeric mixture.

General Procedure C (Diborylation)

General procedure A was applied with 2.5-3 equivalents of HBPin.

Borylation with Phosphine Ligands

General Procedure D

In a glove box, (Ind)Ir(COD) (8.3 mg, 2.00 mmol, 2.00 mol % Ir) and dmpe (3 mg, 2.00 mmol, 2.00 mol %) or dppe (8 mg, 2.00 mmol, 2.00 mol %) were weighed in two separate test tubes. HBPin (218 μL, 190 mg, 1.50 mmol, 1.50 equiv) was added to the dmpe test tube and the resulting solution was than mixed with (Ind)Ir(COD). This catalyst solution was added to a Schlenk flask equipped with a magnetic stirring bar. n-Octane (3×1 mL) was used to wash the test tubes and washings were also transferred to the Schlenk flask. The Schlenk flask was closed, brought out of the glove box, and was attached to Schlenk line. It was heated at elevated temperature under N₂. The reaction was monitored by GC-FID/MS. After completion of the reaction, the volatile materials were removed on a rotary evaporator. The crude material was dissolved in CH₂Cl₂ and passed through a short plug of silica to afford the corresponding borylated product.

### i. MonoBorylation of 2-Substituted Thiophenes

### a. Borylation of 2-Iodothiophene

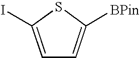

The general procedure A was applied to 2-iodothiophene (111 μL, 210 mg, 1 mmol, 1 equiv) and HBPin (218 μL, 192 mg, 1.50 mmol, 1.50 equiv) for 1 h. The product was isolated as a white solid (310 mg, 92% yield, mp 48-49° C.). ¹H NMR (CDCl₃, 500 MHz): δ 7.27 (d, J=3.5 Hz, 1H), 7.25 (d, J=3.5 Hz, 1H), 1.31 (br s, 12H, 4 CH₃ of BPin); ¹³C NMR {¹H} (CDCl₃, 125 MHz): δ 138.5 (CH), 138.3 (CH), 84.3 (2 C), 81.5 (C), 24.7 (4 CH₃ of BPin); ¹¹B NMR (CDCl₃, 96 MHz): δ 28.7; FT-IR (neat) \tilde{ν}ₘₐₓ: 2978, 2932, 1522, 1418, 1314, 1267, 1142, 1064, 1018, 853, 663 cm⁻¹; GC-MS (EI) m/z (% relative intensity): M⁺ 336 (100), 321 (13), 250 (6), 236 (14), 209 (12), 167 (43); Anal. Calcd for C₁₀H₁₄BIO₂S: C, 35.75; H, 4.20. Found: C, 36.04; H, 4.24.

### b. Borylation of 2-Acetylthiophene

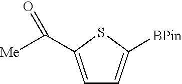

The general procedure A was applied to 2-acetylthiophene (108 μL, 126 mg, 1 mmol, 1 equiv) and HBPin (175 μL, 154 mg, 1.20 mmol, 1.20 equiv) for 0.5 h. The product was isolated as a white solid (213 mg, 85% yield, mp 64-66° C.). ¹H NMR (CDCl₃, 500 MHz): δ 7.69 (d, J=3.8 Hz, 1H), 7.54 (d, J=3.8 Hz, 1H), 2.53 (s, 3H, COCH₃), 1.31 (br s, 12H, 4 CH₃ of BPin); ¹³C NMR {¹H} (CDCl₃, 125 MHz): δ 190.6 (C═O), 149.4 (C), 137.2 (CH), 132.6 (CH), 84.6 (2 C), 27.4 (COCH₃), 24.7 (4 CH₃ of BPin); ¹¹B NMR (CDCl₃, 96 MHz): δ 29.2; FT-IR (neat) \tilde{ν}ₘₐₓ. 2980, 2934, 1669, 1520, 1348, 1288, 1267, 1142, 1020, 852, 667 cm⁻¹; GC-MS (EI) m/z (% relative intensity): M⁺ 252 (77), 237 (100), 209 (15), 195 (8), 179 (5), 166 (33), 153 (14) 137 (12) 109 (6); Anal. Calcd for C₁₂H₁₇BO₃S: C, 57.16; H, 6.80. Found: C, 56.88; H, 7.06.

### c. Borylation of Methyl-2-thiophenecarboxylate

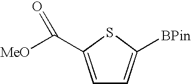

The general procedure A was applied to methyl-2-thiophenecarboxylate (116 μL, 142 mg, 1 mmol, 1 equiv) and HBPin (192 μL, 218 mg, 1.50 mmol, 1.50 equiv) for 0.5 h. The product was isolated as a white solid (252 mg, 94% yield). ¹H NMR (CDCl₃, 500 MHz): δ 7.79 (d, J=3.7 Hz, 1H), 7.53 (d, J=3.7 Hz, 1H), 3.87 (s, 3H, CO₂CH₃), 1.33 (br s, 12H, 4 CH₃ of BPin); ¹³C NMR {¹H} (CDCl₃, 125 MHz): δ 162.6 (C═O), 139.4 (C), 136.9 (CH), 133.9 (CH), 84.6 (2 C), 52.2 (CO₂CH₃), 24.7 (4 CH₃ of BPin); ¹¹B NMR (CDCl₃, 96 MHz): δ 29.1; FT-IR (neat) \tilde{ν}ₘₐₓ: 2970, 1719, 1527, 1354, 1248, 1145, 1097, 852, 832, 752, 665 cm⁻¹; GC-MS (EI) m/z (% relative intensity): M⁺ 268 (71), 253 (91), 237 (56), 182 (100); Anal. Calcd for C₁₂H₁₇BO₄S: C, 53.75; H, 6.39. Found: C, 53.44; H, 6.44.

### d. Borylation of 2-trimethylsilylthiophene

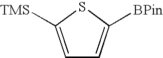

The general procedure A was applied to 2-trimethylsilylthiophene (312 mg, 2 mmol, 1 equiv) and HBPin (435 μL, 384 mg, 3.00 mmol, 1.50 equiv) for 30 minutes. The product was isolated as a white solid (523 mg, 93% yield, mp 61-62° C.). ¹H NMR (CDCl₃, 500 MHz): δ 7.67 (d, J=3.3 Hz, 1H), 7.31 (d, J=3.3 Hz, 1H), 1.32 (br s, 12H, 4 CH₃ of BPin), 0.30 (s, 9H, 3 CH₃ of TMS); ¹³C NMR {¹H} (CDCl₃, 75 MHz): δ 148.4 (C), 137.8 (CH), 135.0 (CH), 84.0 (2 C), 24.8 (4 CH₃ of BPin), −0.1 (3 CH₃ of TMS); ¹¹B NMR (CDCl₃, 96 MHz): δ 29.6; FT-IR (neat) \tilde{ν}ₘₐₓ: 3054, 2980, 2957, 1514, 1435, 1346, 1331, 1259, 1250, 1142, 1072, 981, 841, 821, 758, 699 cm⁻¹; GC-MS (EI) m/z (% relative intensity): M⁺ 282 (14), 267 (100), 239 (31), 167 (8); Anal. Calcd for C₁₃H₂₃BO₂SSi: C, 55.31; H, 8.21. Found: C, 54.85; H, 8.74; HRMS (EI): m/z 282.1285 [(M⁺); Calcd for C₁₃H₂₃BO₂SSi: 282.1281].

### e. Borylation of 2-chlorothiophene

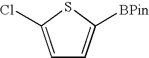

The general procedure A was applied to 2 chlorothiophene (184 μL, 236 mg, 2 mmol, 1 equiv) and HBPin (348 μL, 307 mg, 2.40 mmol, 1.20 equiv) for 15 minutes. The product was isolated as colorless oil (476 mg, 97% yield). ¹H NMR (CDCl₃, 500 MHz): δ 7.37 (d, J=3.7 Hz, 1H), 6.95 (d, J=3.7 Hz, 1H), 1.31 (br s, 12H, 4 CH₃ of BPin); ¹³C NMR {¹H} (CDCl₃, 75 MHz): δ 136.8 (C), 136.7 (CH), 127.6 (CH), 84.3 (2 C), 24.7 (4 CH₃ of BPin); ¹¹B NMR (CDCl₃, 96 MHz): δ 28.8; FT-IR (neat) \tilde{ν}ₘₐₓ: 2980, 2932, 1530, 1433, 1352, 1334, 1282, 1271, 1142, 1035, 852, 804, 663 cm⁻¹; GC-MS (EI) m/z (% relative intensity): M⁺ 244 (100), 229 (17), 201 (21), 184 (17) 158 (12); Anal. Calcd for C₁₀H₁₄BClO₂S: C, 49.11; H, 5.77. Found: C, 49.12; H, 5.98.

### f. Borylation of 2-methoxythiophene

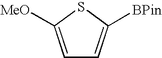

The general procedure A was applied to 2-methoxythiophene (202 μL, 228 mg, 2 mmol, 1 equiv) and HBPin (348 μL, 307 mg, 2.40 mmol, 1.20 equiv) for 1 h. The product was isolated as colorless oil (395 mg, 82% yield). ¹H NMR (CDCl₃, 500 MHz): δ 7.31 (d, J=3.8 Hz, 1H), 6.28 (d, J=3.8 Hz, 1H), 3.89 (s, 3H, OCH₃), 1.31 (br s, 12H, 4 CH₃ of BPin); ¹³C NMR {¹H} (CDCl₃, 125 MHz): δ 172.8 (C), 136.5 (CH), 106.1 (CH), 84.8 (2 C), 60.3 (OCH₃), 24.7 (4 CH₃ of BPin); ¹¹B NMR (CDCl₃, 96 MHz): δ 29.0; FT-IR (neat) \tilde{ν}ₘₐₓ: 3084, 2978, 2934, 2870, 1549, 1483, 1423, 1365, 1302, 1213, 1143, 989, 854, 781, 684, 661 cm⁻¹; GC-MS (EI) m/z (% relative intensity): M⁺ 240 (100), 225 (5), 197 (12), 180 (18); Anal. Calcd for C₁₁H₁₇BO₃S: C, 55.02; H, 7.14. Found: C, 54.72; H, 7.60.

### g. Borylation of 2-Chloro-3-bromothiophene

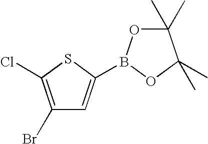

The general procedure A was applied to 2-chloro-3-bromothiophene (110 μL, 197 mg, 1 mmol, 1 equiv) and HBPin (192 μL, 218 mg, 1.50 mmol, 1.50 equiv) for 10 minutes. The product was isolated as a white solid (253 mg, 78% yield, mp 60-61° C.). ¹H NMR (CDCl₃, 500 MHz): δ 7.38 (s, 1H), 1.30 (br s, 12H, 4 CH₃ of BPin); ¹³C NMR {¹H} (CDCl₃, 125 MHz): δ 138.9 (CH), 133.2 (C), 112.0 (C), 84.6 (2 C), 24.7 (4 CH₃ of BPin); ¹¹B NMR (CDCl₃, 96 MHz): δ 28.5; FT-IR (neat) \tilde{ν}ₘₐₓ: 2980, 2932, 1523, 1425, 1340, 1267, 1142, 1041, 852, 661 cm⁻¹; GC-MS (EI) m/z (% relative intensity): M⁺ 324 (100), 322 (73), 309 (45), 281 (26), 264 (29), 243 (38); Anal. Calcd for C₁₀H₁₃BBrClO₂S: C, 37.13; H, 4.05. Found: C, 37.20; H, 4.16.

Note: Attempted borylation of 2,5-dichloro-3-bromo-thiophene with borylation procedure D also gave the same product where C—Cl bond was borylated and the single monoborylated product was isolated in 73% yield (see attempted monoborylation of tri-substituted thiophene). Only one of the two C—Cl bonds is activated with chemoselectivity greater than 99%. The NMR data matched with the borylated product of 2-Cl-3-Br-thiophene as described above.

### ii. Monoborylation of 3-Substituted Thiophenes

### a. Borylation of 3-Cyanothiophene

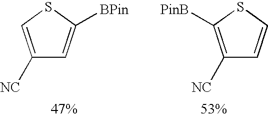

The general procedure B was applied to 3-cyanothiophene (182 μL, 218 mg, 2.00 mmol, 2.00 equiv) and HBPin (145 μL, 128 mg, 1 mmol, 1 equiv) for 1 h. The ratio of two monoborylated products at the end of reaction was 47:53 by GC-FID. The monoborylated product mixture was isolated as a white solid (126 mg, 54% yield). ¹H NMR (CDCl₃, 300 MHz): δ (major isomer) 7.62 (d, J=4.9 Hz, 1H), 7.38 (d, J=4.9 Hz, 1H), 1.36 (br s, 12H, CH₃ of BPin), (minor isomer) 8.13 (d, J=1.2 Hz, 1H), 7.75 (d, J=1.2 Hz, 1H), 1.33 (br s, 12H, 4 CH₃ of BPin); ¹³C NMR {¹H} (CDCl₃, 125 MHz): δ (major isomer) 132.7 (CH), 131.4 (CH), 118.3 (C), 115.2 (C), 84.9 (2 C), 24.7 (4 CH₃ of BPin), (minor isomer) 140.8 (CH), 138.1 (CH), 114.7 (C), 111.9 (C), 85.1 (2 C), 24.7 (4 CH₃ of BPin); ¹¹B NMR (CDCl₃, 96 MHz): δ 28.6; FT-IR (neat) \tilde{ν}ₘₐₓ: 2980, 2231, 1429, 1319, 1142, 1039, 850, 628 cm⁻¹; GC-MS (EI) m/z (% relative intensity): (major isomer) M⁺¹ 236 (100), 220 (78), 194 (51), 178 (33), 149 (36), 136 (31), (minor isomer) M⁺ 235 (7), 220 (100), 192 (9), 149 (37), 136 (15); Anal. Calcd for C₁₁H₁₄BNO₂S: C, 56.19; H, 6.0; N, 5.96. Found: C, 55.74; H, 5.99; N, 6.0.

### b. Monoborylation of 3-Chlorothiophene

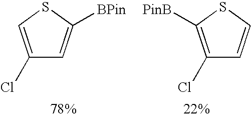

The general procedure B was applied to 3-chlorothiophene (186 μL, 237 mg, 2.00 mmol, 2.00 equiv) and HBPin (145 μL, 128 mg, 1 mmol, 1 equiv) for 1 h. The ratio of two monoborylated products at the end of reaction was 78:22 by GC-FID. The monoborylated product mixture was isolated as a white solid (160 mg, 66% yield). ¹H NMR (CDCl₃, 300 MHz): δ (major isomer) 7.43 (d, J=1.0 Hz, 1H), 7.35 (d, J=1.0 Hz, 1H), 1.32 (br s, 12H, 4 CH₃ of BPin), (minor isomer) 7.51 (d, J=5.0 Hz, 1H), 7.01 (d, J=5.0 Hz, 1H), 1.34 (br s, 12H, 4 CH₃ of BPin); ¹³C NMR {¹H} (CDCl₃, 125 MHz): δ (major isomer) 136.9 (CH), 131.8 (C), 126.7 (CH), 84.4 (2 C), 24.7 (4 CH₃ of BPin); ¹¹B NMR (CDCl₃, 96 MHz): δ 29.0; FT-IR (neat) \tilde{ν}ₘₐₓ: 3107, 2980, 2932, 1522, 1421, 1356, 1336, 1142, 1026, 854, 665 cm⁻¹; GC-MS (EI) m/z (% relative intensity): M⁺ 244 (100), 246 (38), 231 (15), 229 (38), 209 (24), 158 (27); Anal. Calcd for C₁₀H₁₄BClO₂S: C, 49.11; H, 5.77. Found: C, 49.33; H, 5.81.

### c. Monoborylation of 3-Bromothiophene

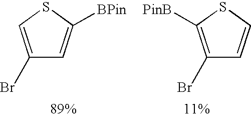

The general procedure B was applied to 3-bromothiophene (190 μL, 326 mg, 2.00 mmol, 2.00 equiv) and HBPin (145 μL, 128 mg, 1 mmol, 1 equiv) for 1 h. The ratio of two monoborylated products at the end of reaction was 89:11 by GC-FID. The monoborylated product mixture was isolated as a white solid (209 mg, 72% yield). ¹H NMR (CDCl₃, 300 MHz): δ (major isomer) 7.49 (d, J=1.2 Hz, 1H), 7.46 (d, J=1.2 Hz, 1H), 1.32 (br s, 12H, 4 CH₃ of BPin), (minor isomer) 7.48 (d, J=5.0 Hz, 1H), 7.08 (d, J=5.0 Hz, 1H), 1.34 (br s, 12H, 4 CH₃ of BPin); ¹³C NMR {¹H} (CDCl₃, 125 MHz): δ (major isomer) 139.3 (CH), 129.5 (CH), 111.2 (C), 84.4 (2 C), 24.7 (4 CH₃ of BPin); ¹¹B NMR (CDCl₃, 96 MHz): δ 29.0; FT-IR (neat) \tilde{ν}ₘₐₓ: 2980, 1518, 1415, 1350, 1143, 1026, 852, 665 cm⁻¹; GC-MS (EI) m/z (% relative intensity): (major isomer) M⁺ 289 (51), 290 (98), 288 (100), 275 (61), 273 (55), 247 (18), 245 (21), 230 (19) 204 (41), (minor isomer) M⁺ 289 (13), 290 (25), 288 (27), 275 (10), 273 (9), 209 (100), 189 (11), 167 (67); Anal. Calcd for C₁₀H₁₄BBrO₂S: C, 41.56; H, 4.88. Found: C, 41.74; H, 4.88.

### d. Monoborylation of 3-Methylthiophene

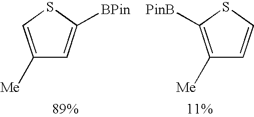

The general procedure B was applied to 3-Methylthiophene (194 μL, 196 mg, 2.00 mmol, 2.00 equiv) and HBPin (145 μL, 128 mg, 1 mmol, 1 equiv) for 1 h. The ratio of two monoborylated products at the end of reaction was 89:11 by GC-FID. The monoborylated product mixture was isolated as a white solid (X mg, Y % yield). ¹H NMR (CDCl₃, 300 MHz): δ (major isomer) 7.42 (d, J=0.7 Hz, 1H), 7.17 (t, J=1.1 Hz, 1H), 2.27 (d J=0.5 Hz, 1H), 1.32 (br s, 12H, 4 CH₃ of BPin), (minor isomer) 7.46 (d, J=4.6 Hz, 1H), 6.95 (d, J=4.6 Hz, 1H), 2.47 (s, 1H), 1.30 (br s, 12H, 4 CH₃ of BPin); ¹³C NMR {¹H} (CDCl₃, 125 MHz): δ (major isomer) 139.4 (CH), 138.9 (C), 128.0 (CH), 83.9 (2 C), 24.7 (4 CH₃ of BPin), 14.9 (CH₃); ¹¹B NMR (CDCl₃, 96 MHz): δ 29.5; FT-IR (neat) \tilde{ν}ₘₐₓ: 2978, 2930, 1550, 1441, 1371, 1327, 1302, 1271, 1143, 1028, 962, 854 665 cm⁻¹; GC-MS (EI) m/z (% relative intensity): (major isomer) M⁺ 224 (100), 209 (27), 181 (18), 138 (44), (minor isomer) M⁺ 224 (100), 209 (68), 167 (64), 138 (54), 124 (61); Anal. Calcd for C₁₁H₁₇BO₂S: C, 58.95; H, 7.65. Found: C, 58.65; H, 8.09.

### e. Borylation of 3-trimethylsilylthiophene

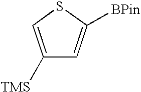

The general procedure A was applied to 3-trimethylsilylthiophene (156 mg, 1 mmol, 1 equiv) and HBPin (174 μL, 154 mg, 1.20 mmol, 1.20 equiv) for 30 minutes. The product was isolated as a white solid (222 mg, 79% yield, mp 87-89° C.). ¹H NMR (CDCl₃, 300 MHz): δ 7.71 (d, J=1.0 Hz, 1H), 7.69 (d, J=1.0 Hz, 1H), 1.33 (br s, 12H, 4 CH₃ of BPin), 0.24 (s, 9H, 3 CH₃ of TMS); ¹³C NMR {¹H} (CDCl₃, 75 MHz): δ 142.4 (C), 141.8 (CH), 138.4 (CH), 83.8 (2 C), 24.6 (4 CH₃ of BPin), −0.6 (3 CH₃ of TMS); ¹¹B NMR (CDCl₃, 96 MHz): δ 29.5; FT-IR (neat) \tilde{ν}ₘₐₓ: 2980, 2955, 1510, 1410, 1325, 1263, 1250, 1143, 1105, 1028, 902, 852, 839, 754, 667 cm⁻¹; GC-MS (EI) m/z (% relative intensity): M⁺ 282 (7), 267 (100), 239 (2), 167 (7); Anal. Calcd for C₁₃H₂₃BO₂SSi: C, 55.31; H, 8.21. Found: C, 54.68; H, 8.47; HRMS (EI): m/z 282.1283 [(M⁺); Calcd for C₁₃H₂₃BO₂SSi: 282.1281].

### f. Borylation of 3-acetylthiophene

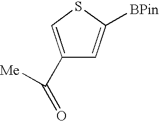

The general procedure A was applied to 3-acetylthiophene (126 mg, 1 mmol, 1 equiv) and HBPin (174 μL, 154 mg, 1.20 mmol, 1.20 equiv) for 15 minutes. The product was isolated as colorless oil (206 mg, 82% yield). ¹H NMR (CDCl₃, 500 MHz): δ 8.26 (d, J=1.1 Hz, 1H), 8.00 (d, J=1.1 Hz, 1H), 2.50 (s, 3H, COCH₃) 1.32 (br s, 12H, 4 CH₃ of BPin); ¹³C NMR {¹H} (CDCl₃, 125 MHz): δ 192.0 (C═O), 143.8 (C), 138.1 (CH), 137.0 (CH), 84.5 (2 C), 27.8 (COCH₃), 24.8 (4 CH₃ of BPin); ¹¹B NMR (CDCl₃, 96 MHz): δ 29.2; FT-IR (neat) \tilde{ν}ₘₐₓ: 3098, 2980, 2934, 1680, 1530, 1448, 1381, 1373, 1340, 1305, 1215, 1143, 1024, 850, 667 cm⁻¹; GC-MS (EI) m/z (% relative intensity): M⁺ 252 (21), 237 (55), 209 (100), 195 (9), 153 (22), 137 (19); Anal. Calcd for C₁₂H₁₇BO₃S: C, 57.16; H, 6.80. Found: C, 56.77; H, 7.19.

### g. Borylation of methyl 3-thiophenecarboxylate

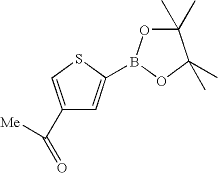

The general procedure A was applied to methyl 3-thiophenecarboxylate (121 μL, 142 mg, 1 mmol, 1 equiv) and HBPin (174 μL, 154 mg, 1.20 mmol, 1.20 equiv) for 1 h. The product was isolated as a white solid (256 mg, 95% yield, mp 84-85° C.). ¹H NMR (CDCl₃, 500 MHz): δ 8.31 (d, J=1.0 Hz, 1H), 8.01 (d, J=1.0 Hz, 1H), 3.84 (s, 3H, CO₂CH₃) 1.33 (br s, 12H, CH₃ of BPin); ¹³C NMR {¹H} (CDCl₃, 75 MHz): δ 163.1 (C═O), 138.8 (CH), 137.9 (CH), 134.9 (C), 84.4 (2 C), 51.6 (CO₂CH₃), 24.7 (4 CH₃ of BPin); ¹¹B NMR (CDCl₃, 96 MHz): δ 29.4; FT-IR (neat) \tilde{ν}ₘₐₓ: 3107, 2980, 2951, 1722, 1537, 1458, 1431, 1388, 1373, 1336, 1307, 1224, 1143, 1024, 987, 852, 752, 667 cm⁻¹; GC-MS (EI) m/z (% relative intensity): M⁺ 268 (65), 253 (100), 237 (22), 225 (39), 211 (29), 193 (12), 182 (45), 169 (41), 137 (27); Anal. Calcd for C₁₂H₁₇BO₄S: C, 53.75; H, 6.39. Found: C, 53.54; H, 6.66.

### h. Borylation of 3-p-tolylthiophene

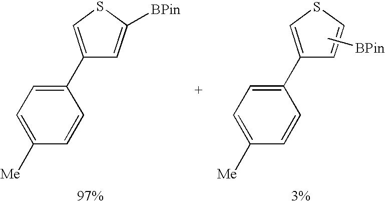

The general procedure B was applied to 3-p-tolylthiophene (192 mg, 1.1 mmol, 1.1 equiv) and HBPin (145 μL, 128 mg, 1.00 mmol, 1.00 equiv) for 1 h. The ratio of two monoborylated isomers at the end of reaction was 97:3 by GC-FID. The product was isolated as colorless oil (223 mg, 74% yield). ¹H NMR (CDCl₃, 300 MHz): δ 7.91 (d, J=1.2 Hz, 1H), 7.68 (d, J=1.2 Hz, 1H), 7.48-7.52 (m, 2H), 7.17-7.20 (m, 2H), 2.35 (s, 3H, CH₃) 1.36 (br s, 12H, 4 CH₃ of BPin); ¹³C NMR {¹H} (CDCl₃, 75 MHz): δ 143.8 (C), 136.8 (C), 136.2 (CH), 132.9 (C), 129.5 (CH), 126.9 (CH), 126.4 (CH), 84.2 (2 C), 24.8 (4 CH₃ of BPin), 21.1 (CH₃); ¹¹B NMR (CDCl₃, 96 MHz): δ 29.4; FT-IR (neat) \tilde{ν}ₘₐₓ: 3090, 2978, 2928, 1547, 1441, 1379, 1371, 1329, 1311, 1269, 1143, 1026, 850, 819, 771, 667 cm⁻¹; GC-MS (EI) m/z (% relative intensity): M⁺ 300 (100), 285 (12), 214 (12); Anal. Calcd for C₁₇H₂₁BO₂S: C, 68.01; H, 7.05. Found: C, 68.54; H, 6.97; HRMS (EI): m/z 300.1360 [(M⁺); Calcd for C₁₇H₂₁BO₂S: 300.1355].

### iii. Monoborylation of 2,5-Disubstituted Thiophenes

### a. Borylation of 2-5-Dichlorothiophene

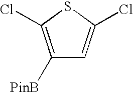

The general procedure A was applied to 2-5-dichlorothiophene (107 μL, 153 mg, 1 mmol, 1 equiv) and HBPin (218 μL, 192 mg, 1.50 mmol, 1.50 equiv) for 20 h. The product was isolated as a white solid (240 mg, 86% yield, mp 35-36° C.). ¹H NMR (CDCl₃, 500 MHz): δ 6.94 (s, 1H), 1.30 (br s, 12H, 4 CH₃ of BPin); ¹³C NMR {¹H} (CDCl₃, 125 MHz): δ 137.1 (C), 131.1 (CH), 126.2 (C), 84.0 (2 C), 24.8 (4 CH₃ of BPin); ¹¹B NMR (CDCl₃, 96 MHz): δ 28.5; FT-IR (neat) \tilde{ν}ₘₐₓ: 2980, 1535, 1437, 1371, 1313, 1263, 1142, 1032, 966, 889, 848, 692 cm⁻¹; GC-MS (EI) m/z (% relative intensity): M⁺ 278 (100), 280 (68), 263 (32), 265 (22), 243 M-35 (79), 245 (30), 201 (51); Anal. Calcd for C₁₀H₁₃BCl₂O₂S: C, 43.05; H, 4.70. Found: C, 43.26; H, 4.74.

### b. Borylation of 2-5-Dibromothiophene

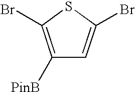

The general procedure A was applied to 2-5-dibromothiophene (113 μL, 142 mg, 1 mmol, 1 equiv) and HBPin (218 μL, 192 mg, 1.50 mmol, 1.50 equiv) with 6% [Ir] catalyst loading for 36 h. Additional 3% [Ir] and 1 equiv of HBPin was added at this stage and the reaction was run for 12 more h at room temperature. The ratio of the starting material to product after 48 h was 11:89. The product was isolated as a white solid (206 mg, 56% yield, mp 72-73° C.). ¹H NMR (CDCl₃, 500 MHz): δ 7.09 (s, 1H), 1.31 (br s, 12H, 4 CH₃ of BPin); ¹³C NMR {¹H} (CDCl₃, 125 MHz): δ 135.8 (CH), 121.9 (C), 110.9 (C), 84.0 (2 C), 24.8 (4 CH₃ of BPin); ¹¹B NMR (CDCl₃, 96 MHz): δ 28.5; FT-IR (neat) \tilde{ν}ₘₐₓ: 2978, 1525, 1123, 1365, 1307, 1248, 1143, 991, 962, 883, 848, 690 cm⁻¹; GC-MS (EI) m/z (% relative intensity): M⁺ 368 (100), 370 (51), 366 (52), 353 (18), 287 (56), 289 (59), 268 (28), 208 (77), 166 (69); Calcd for C₁₀H₁₃BBr₂O₂S: C, 32.65; H, 3.56. Found: C, 32.92; H, 3.57.

### c. Borylation of 2-5-Dimethylthiophene

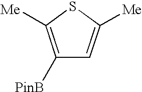

The general procedure D was applied to 2-5-methylthiophene (228 μL, 224 mg, 2 mmol, 1 equiv) and neat HBPin (435 μL, 384 mg, 3.00 mmol, 1.50 equiv) with 2% [Ir] catalyst loading at 150° C. for 16 h. The product was isolated as a colorless semi solid (460 mg, 97% yield). ¹H NMR (CDCl₃, 300 MHz): δ 6.81 (d, J=1.2 Hz, 1H), 2.59 (s, 3H, CH₃), 2.38 (d, J=0.4 Hz, 3H, CH₃), 1.30 (br s, 12H, 4 CH₃ of BPin); ¹³C NMR {¹H} (CDCl₃, 125 MHz): δ 150.8 (C), 136.1 (C), 130.7 (CH), 83.0 (2 C), 24.8 (4 CH₃ of BPin), 15.6 (CH₃), 14.7 (CH₃); ¹¹B NMR (CDCl₃, 96 MHz): δ 29.3; FT-IR (neat) \tilde{ν}ₘₐₓ: 2978, 2924, 1493, 1394, 1304, 1265, 1145, 868, 700 cm⁻¹; GC-MS (EI) m/z (% relative intensity): M⁺ 238 (100), 223 (8), 181 (37); Anal. Calcd for C₁₂H₁₉BO₂S: C, 60.52; H, 8.04. Found: C, 60.62; H, 8.18.

### d. Borylation of 2-Bromo-5-chlorothiophene

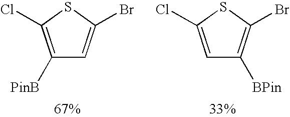

The general procedure A was applied to 2-bromo-5-chlorothiophene (110 μL, 197 mg, 1 mmol, 1 equiv) and HBPin (290 μL, 256 mg, 2.00 mmol, 2.00 equiv) with 6% [Ir] catalyst loading for 20 h. The ratio of two monoborylated products at the end of reaction was 67:33 by GC-FID. The monoborylated product mixture was isolated as a white solid (281 mg, 87% yield). ¹H NMR (CDCl₃, 500 MHz): δ (major isomer) 7.10 (s, 1H), 1.30 (br s, 12H, 4 CH₃ of BPin), (minor isomer) 6.94 (s, 1H), 1.30 (br s, 12H, 4 CH₃ of BPin); ¹³C NMR {¹H} (CDCl₃, 125 MHz): δ (major isomer) 139.6 (C), 134.9 (CH), 108.3 (C), 84.0 (2C), 24.8 (4 CH₃ of BPin), (minor isomer) 132.0 (CH), 128.9 (C), 119.5 (C), 84.1 (2 C), 24.8 (4 CH₃ of BPin); ¹¹B NMR (CDCl₃, 96 MHz): δ 28.5; FT-IR (neat) 2980, 1527, 1427, 1371, 1253, 1140, 1028, 962, 848, 693 \tilde{ν}ₘₐₓ: cm⁻¹; GC-MS (EI) m/z (% relative intensity): (major isomer) M⁺ 324 (100), 322 (78), 289 (67), 287 (64), 208 (40), 166 (34), (minor isomer) M⁺ 324 (89), 322 (69), 309 (23), 245 (41), 243 (99), 203 (43), 201 (100), 166 (50); Anal. Calcd for C₁₀H₁₃BBrClO₂S: C, 37.13; H, 4.05. Found: C, 37.25; H, 4.05.

Note: The data for the pure major isomer is described elsewhere in this supporting information.

### e. Borylation of 2-Chloro-5-iodothiophene

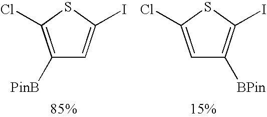

The general procedure A was applied to 2-chloro-5-iodothiophene (122 mg, 0.5 mmol, 1 equiv) and HBPin (109 μL, 96 mg, 0.75 mmol, 1.50 equiv) for 20 h. The ratio of two monoborylated products at the end of reaction was 85:15 by GC-FID. The monoborylated product mixture was isolated as a white solid (165 mg, 89% yield). ¹H NMR (CDCl₃, 300 MHz): δ (major isomer) 7.31 (s, 1H), 1.30 (br s, 12H, 4 CH₃ of BPin), (minor isomer) 6.87 (s, 1H), 1.31 (br s, 12H, 4 CH₃ of BPin); ¹³C NMR {¹H} (CDCl₃, 125 MHz): δ (major isomer) 143.4 (C), 142.3 (CH), 84.0 (2 C), 69.3 (C), 24.8 (4 CH₃ of BPin), (minor isomer) 132.8 (CH), 84.2 (2 C), 81.1 (C), 24.8 (4 CH₃ of BPin); ¹¹B NMR (CDCl₃, 96 MHz): δ 28.3; FT-IR (neat) \tilde{ν}ₘₐₓ: 2978, 1523, 1414, 1371, 1248, 1140, 1024, 966, 881, 848 690 cm⁻¹; GC-MS (EI) m/z (% relative intensity): (major isomer) M⁺ 370 (100), 355 (13), 335 (29), 270 (25), 208 (15), 166 (11), (minor isomer) M⁺ 370 (100), 355 (10), 270 (24), 243 (13), 201 (32), 166 (21); Anal. Calcd for C₁₀H₁₃BIClO₂S: C, 32.42; H, 3.54. Found: C, 32.58; H, 3.38.

### f. Borylation of 2-Chloro-5-methylthiophene

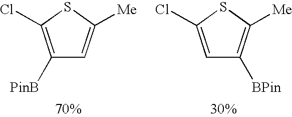

The general procedure A was applied to 2-chloro-5-methylthiophene (133 mg, 1 mmol, 1 equiv) and HBPin (218 μL, 192 mg, 1.50 mmol, 1.50 equiv) for 18 h. The ratio of two monoborylated products at the end of reaction was 70:30 by GC-FID. The monoborylated product mixture was isolated as a colorless semi solid (221 mg, 86% yield). ¹H NMR (CDCl₃, 300 MHz): δ (major isomer) 6.77 (q, J=1.2 Hz, 1H), 2.35 (d, J=1.2 Hz, 3H, CH₃), 1.31 (br s, 12H, 4 CH₃ of BPin), (minor isomer) 6.95 (s, 1H), 2.60 (s, 3H, CH₃), 1.28 (br s, 12H, 4 CH₃ of BPin); ¹³C NMR {¹H} (CDCl₃, 125 MHz): δ (major isomer) 137.4 (C), 137.0 (C), 130.1 (CH), 83.6 (2 C), 24.8 (4 CH₃ of BPin), 14.9 (CH₃), (minor isomer) 151.1 (C), 131.6 (CH), 125.4 (C), 83.4 (2 C), 24.8 (4 CH₃ of BPin), 15.7 (CH₃); ¹¹B NMR (CDCl₃, 96 MHz): δ 29.1; FT-IR (neat) \tilde{ν}ₘₐₓ: 2980, 2926, 1556, 1475, 1390, 1371, 1309, 1257, 1143, 1026, 966, 898, 850, 696 cm⁻¹; GC-MS (EI) m/z (% relative intensity): M⁺; Anal. Calcd for C₁₁H₁₆BClO₂S: C, 51.10; H, 6.24. Found: C, 51.66; H, 6.58; HRMS (EI): m/z 258.0653 [(M⁺); Calcd for C₁₁H₁₆BClO₂S: 258.06526].

### g. Borylation of 2-Chloro-5-trimethylsilylthiophene

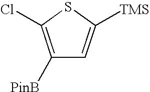

The general procedure A was applied to 2-chloro-5-trimethylsilylthiophene (382 mg, 2 mmol, 1 equiv) and HBPin (435 μL, 384 mg, 3.00 mmol, 1.50 equiv) for 6 h. The single monoborylated product was isolated as a solid (589 mg, 93% yield, mp 68-69° C.). ¹H NMR (CDCl₃, 500 MHz): δ 7.26 (s, 1H), 1.32 (br s, 12H, 4 CH₃ of BPin), 0.26 (s, 9H, 3 CH₃ of TMS); ¹³C NMR {¹H} (CDCl₃, 125 MHz): δ 144.7 (C), 139.42 (CH), 139.37 (C), 83.7 (2 C), 24.8 (4 CH₃ of BPin), −0.24 (3 CH₃ of TMS); ¹¹B NMR (CDCl₃, 96 MHz): δ 29.1; FT-IR (neat) \tilde{ν}ₘₐₓ: 2980, 1525, 1415, 1363, 1307, 1253, 1238, 1143, 993, 841, 758, 696 cm⁻¹; GC-MS (EI) m/z (% relative intensity) 316 (33), 301 (100), 281 (6), 201 (15): M⁺; Anal. Calcd for C₁₃H₂₂BClO₂SSi: C, 49.30; H, 7.00. Found: C, 49.16; H, 7.16.

Diborylation.

### iv. Diborylation of 2-Substituted Thiophenes

### a. Diborylation of 2-Cyanothiophene

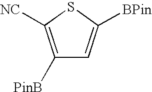

The general procedure C was applied to 2-cyanoothiophene (94 μL, 109 mg, 1 mmol, 1 equiv) and HBPin (435 μL, 384 mg, 3.00 mmol, 3.00 equiv) for 1 h. The single diborylated product was isolated as a white solid (317 mg, 88% yield, mp 132-133° C.). ¹H NMR (CDCl₃, 500 MHz): δ 7.87 (s, 1H), 1.33 (br s, 12H, CH₃ of BPin), 1.31 (s, 12H, 4 CH₃ of BPin); ¹³C NMR {¹H} (CDCl₃, 125 MHz): δ 143.1 (CH), 123.2 (C), 114.3 (C), 84.8 (2 C), 84.6 (2 C), 24.8 (4 CH₃ of BPin), 24.7 (4 CH₃ of BPin); ¹¹B NMR (CDCl₃, 96 MHz): δ 28.6; FT-IR (neat) \tilde{ν}ₘₐₓ: 2980, 2934, 2220, 1531, 1458, 1373, 1319, 1138, 1030, 966, 848, 667 cm⁻¹; GC-MS (EI) m/z (% relative intensity): M⁺ 361 (73), 346 (53), 320 (100), 303 (31), 275 (24), 262 (29); Anal. Calcd for C₁₇H₂₅B₂NO₄S: C, 56.55; H, 6.98; Found: C, 56.45; H, 7.16.

### b. Diborylation of 2-Chlorothiophene

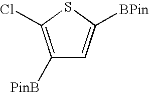

The general procedure C was applied to 2-chlorothiophene (92 μL, 118 mg, 1 mmol, 1 equiv) and HBPin (363 μL, 320 mg, 2.50 mmol, 2.50 equiv) for 12 h. The single diborylated product was isolated as a white solid (315 mg, 85% yield, mp 130-131° C.). ¹H NMR (CDCl₃, 500 MHz): δ 7.72 (s, 1H), 1.30 (br s, 12H, 4 CH₃ of BPin), 1.29 (s, 12H, 4 CH₃ of BPin); ¹³C NMR {¹H} (CDCl₃, 125 MHz): δ 146.3 (C), 143.6 (CH), 84.2 (2 C), 83.8 (2 C), 24.8 (4 CH₃ of BPin), 24.7 (4 CH₃ of BPin); ¹¹B NMR (CDCl₃, 96 MHz): δ 29.0; FT-IR (neat) \tilde{ν}ₘₐₓ: 2976, 2928, 1539, 1456, 1371, 1340, 1309, 1140, 1042, 964, 851, 665 cm⁻¹; GC-MS (EI) m/z (% relative intensity): M⁺ 370 (100), 372 (40), 355 (46), 335 (85), 313 (21), 285 (39), 227 (52); Anal. Calcd for C₁₆H₂₅B₂ClO₄S: C, 51.87; H, 6.80; Found: C, 51.69; H, 7.00.

### c. Diborylation of 2-Bromothiophene

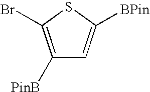

The general procedure C was applied to 2-bromothiophene (97 μL, 163 mg, 1 mmol, 1 equiv) and HBPin (363 μL, 320 mg, 2.50 mmol, 2.50 equiv) for 12 h. The single diborylated product was isolated as a white solid (381 mg, 92% yield, mp 116-118° C.). ¹H NMR (CDCl₃, 500 MHz): δ 7.69 (s, 1H), 1.30 (br s, 12H, 4 CH₃ of BPin), 1.29 (s, 12H, 4 CH₃ of BPin); ¹³C NMR {¹H} (CDCl₃, 125 MHz): δ 144.3 (CH), 129.3 (C), 84.2 (2 C), 83.8 (2 C), 24.8 (4 CH₃ of BPin), 24.7 (4 CH₃ of BPin); ¹¹B NMR (CDCl₃, 96 MHz): δ 28.9; FT-IR (neat) \tilde{ν}ₘₐₓ: 2978, 1537, 1452, 1327, 1140, 1026, 964, 850, 665 cm⁻¹; GC-MS (EI) m/z (% relative intensity): M⁺ 414 (100), 416 (97), 401 (22), 335 (71); Anal. Calcd for C₁₆H₂₅B₂BrO₄S: C, 46.31; H, 6.07. Found: C, 46.39; H, 6.06.

### d. Diborylation of 2-Methylthiophene

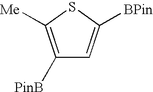

The general procedure C was applied to 2-methylthiophene (97 μL, 98 mg, 1 mmol, 1 equiv) and HBPin (435 μL, 384 mg, 3.00 mmol, 3.00 equiv) for 72 h. The ratio of the two isomeric monoborylated products at the end of reaction was 98.5:1.5 by GC-FID (The GC-FID retention time of the minor diborylated isomer was different from the retention time of the single diborylated product of 3-methylthiophene). The product was isolated as a white solid (316 mg, 90% yield, mp 127-129° C.). ¹H NMR (CDCl₃, 500 MHz): δ 7.81 (s, 1H), 2.68 (s, 3H, CH₃), 1.29 (br s, 12H, 4 CH₃ of BPin), 1.28 (s, 12H, 4 CH₃ of BPin); ¹³C NMR {¹H} (CDCl₃, 125 MHz): δ 159.6 (C), 144.9 (CH), 83.8 (2 C), 83.2 (2 C), 24.9 (4 CH₃ of BPin), 24.7 (4 CH₃ of BPin), 15.9 (CH₃); ¹¹B NMR (CDCl₃, 96 MHz): δ 29.5; FT-IR (neat) \tilde{ν}ₘₐₓ: 2976, 1541, 1475, 1323, 1138, 1012, 844 cm⁻¹; GC-MS (EI) m/z (% relative intensity): M⁺ 350 (87), 335 (32), 293 (100), 264 (45), 250 (38); Anal. Calcd for C₁₇H₂₈B₂O₄S: C, 58.32; H, 8.06. Found: C, 57.96; H, 7.81.

### e. Diborylation of 2-Methoxythiophene

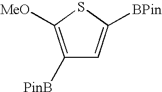

The general procedure C was applied to 2-methylthiophene (101 μL, 114 mg, 1 mmol, 1 equiv) and HBPin (435 μL, 384 mg, 3.00 mmol, 3.00 equiv) for 48 h. The ratio of the two isomeric monoborylated products at the end of reaction was 98.6:1.4 by GC-FID (The GC-FID retention time of the minor diborylated isomer was different from the retention time of the single diborylated product of 3-methoxythiophene). The product was isolated as a white solid (324 mg, 89% yield, mp 110-112° C.). ¹H NMR (CDCl₃, 500 MHz): δ 7.68 (s, 1H), 3.98 (s, 3H, OCH₃), 1.282 (br s, 12H, 4 CH₃ of BPin), 1.280 (s, 12H, 4 CH₃ of BPin); ¹³C NMR {¹H} (CDCl₃, 125 MHz): δ 181.5 (C), 143.8 (CH), 83.7 (2 C), 83.2 (2 C), 61.8 (OCH₃), 24.8 (4 CH₃ of BPin), 24.7 (4 CH₃ of BPin); ¹¹B NMR (CDCl₃, 96 MHz): δ 29.3; FT-IR (neat) \tilde{ν}ₘₐₓ: 2978, 1549, 1481, 1334, 1140, 1022, 968, 852, 663 cm⁻¹; GC-MS (EI) m/z (% relative intensity): M⁺ 366 (100), 352 (10), 324 (5), 282 (11), 250 (13); Anal. Calcd for C₁₇H₂₈B₂O₅S: C, 55.77; H, 7.71. Found: C, 55.41; H, 7.56.

### v. Diborylation of 3-Substituted Thiophenes

### a. Diborylation of 3-Cyanothiophene

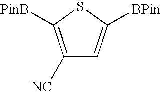

The general procedure C was applied to 3-cyanothiophene (91 μL, 109 mg, 1 mmol, 1 equiv) and HBPin (363 μL, 320 mg, 2.50 mmol, 2.50 equiv) for 0.5 h. The single diborylated product was isolated as a white solid (306 mg, 85% yield, mp 139° C.). ¹H NMR (CDCl₃, 500 MHz): δ 7.80 (s, 1H), 1.34 (br s, 12H, 4 CH₃ of BPin), 1.31 (s, 12H, 4 CH₃ of BPin); ¹³C NMR {¹H} (CDCl₃, 125 MHz): δ 140.3 (CH), 118.8 (C), 115.2 (C), 85.1 (2 C), 84.8 (2 C), 24.7 (8 CH₃ of 2 BPin); ¹¹B NMR (CDCl₃, 96 MHz): δ 28.8; FT-IR (neat) \tilde{ν}ₘₐₓ: 2980, 2936, 2230, 1525, 1373, 1269, 1138, 1055, 962, 850, 667 cm⁻¹; GC-MS (EI) m/z (% relative intensity): M⁺ 361 (70), 346 (45), 331 (28), 320 (100), 304 (80), 275 (39), 262 (51); Anal. Calcd for C₁₇H₂₅B₂NO₄S: C, 56.55; H, 6.98. Found: C, 55.78; H, 6.96; HRMS (FAB): m/z 362.1778 [(M+¹); Calcd for C₁₇H₂₆B₂NO₄S: 362.1768].

### b. Diborylation of 3-Chlorothiophene

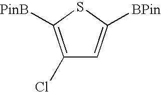

The general procedure C was applied to 3-chlorothiophene (93 μL, 118 mg, 1 mmol, 1 equiv) and HBPin (363 μL, 320 mg, 2.50 mmol, 2.50 equiv) for 1 h. The single diborylated product was isolated as a white solid (337 mg, 91% yield, mp 112-114° C.). ¹H NMR (CDCl₃, 500 MHz): δ 7.45 (s, 1H), 1.32 (br s, 12H, 4 CH₃ of BPin), 1.30 (s, 12H, 4 CH₃ of BPin); ¹³C NMR {¹H} (CDCl₃, 125 MHz): δ 138.3 (CH), 134.7 (C), 84.5 (2 C), 84.3 (2 C), 24.73 (4 CH₃ of BPin), 24.72 (4 CH₃ of BPin); ¹¹B NMR (CDCl₃, 96 MHz): δ 29.1; FT-IR (neat) \tilde{ν}ₘₐₓ: 2980, 1516, 1383, 1348, 1307, 1140, 1041, 958, 853, 669 cm⁻¹; GC-MS (EI) m/z (% relative intensity): M⁺ 370 (57), 355 (38), 335 (100), 285 (40); Anal. Calcd for C₁₆H₂₅B₂ClO₄S: C, 51.87; H, 6.80. Found: C, 51.86; H, 6.88.

### c. Diborylation of 3-Bromothiophene

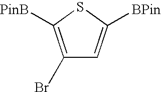

The general procedure C was applied to 3-bromothiophene (95 μL, 163 mg, 1 mmol, 1 equiv) and HBPin (363 μL, 320 mg, 2.50 mmol, 2.50 equiv) for 1 h. The single diborylated product was isolated as a white solid (396 mg, 95% yield, mp 96-98° C.). ¹H NMR (CDCl₃, 500 MHz): δ 7.52 (s, 1H), 1.32 (br s, 12H, 4 CH₃ of BPin), 1.30 (s, 12H, 4 CH₃ of BPin); ¹³C NMR {¹H} (CDCl₃, 125 MHz): δ 141.2 (CH), 119.9 (C), 84.5 (2 C), 84.3 (2 C), 24.73 (4 CH₃ of BPin), 24.71 (4 CH₃ of BPin); ¹¹B NMR (CDCl₃, 96 MHz): δ 28.9; FT-IR (neat) \tilde{ν}ₘₐₓ: 2978, 1510, 1344, 1304, 1269, 1140, 1039, 958, 853, 667 cm⁻¹; GC-MS (EI) m/z (% relative intensity): M⁺ 415 (33), 416 (51), 414 (52), 401 (15), 399 (11), 335 (100), 249 (20), 193 (31); Anal. Calcd for C₁₆H₂₅B₂BrO₄S: C, 46.31; H, 6.07; Found: C, 46.32; H, 6.16.

### d. Diborylation of 3-Methylthiophene

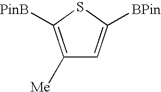

The general procedure C was applied to 3-methylthiophene (97 μL, 98 mg, 1 mmol, 1 equiv) and HBPin (435 μL, 384 mg, 3.00 mmol, 3.00 equiv) for 6 h. The product was isolated as a white solid (268 mg, 77% yield, mp 128-129° C.). ¹H NMR (CDCl₃, 500 MHz): δ 7.43 (s, 1H), 2.43 (s, 3H, CH₃), 1.303 (br s, 12H, 4 CH₃ of BPin), 1.302 (s, 12H, 4 CH₃ of BPin); ¹³C NMR {¹H} (CDCl₃, 125 MHz): δ 149.3 (C), 140.6 (CH), 84.0 (2 C), 83.6 (2 C), 24.8 (4 CH₃ of BPin), 24.7 (4 CH₃ of BPin), 15.6 (CH₃); ¹¹B NMR (CDCl₃, 96 MHz): δ 29.2; FT-IR (neat) \tilde{ν}ₘₐₓ: 2978, 2932, 1537, 1387, 1373, 1332, 1311, 1290, 1267, 1140, 1060, 962, 854, 680, 669 cm⁻¹; GC-MS (EI) m/z (% relative intensity): M⁺ 350 (100), 335 (26), 292 (22), 264 (96) 250 (29); Anal. Calcd for C₁₇H₂₈B₂O₄S: C, 58.32; H, 8.06. Found: C, 58.34; H, 8.45.

### e. Diborylation of 3-p-tolylthiophene

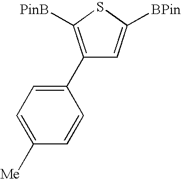

The general procedure C was applied to 3-p-tolylthiophene (174 mg, 1 mmol, 1 equiv) and HBPin (333 μL, 294 mg, 2.30 mmol, 2.30 equiv) for 16 hr. The product was isolated as colorless oil (260 mg, 61% yield). ¹H NMR (CDCl₃, 300 MHz): δ 7.73 (s, 1H), 7.42-7.45 (m, 2H), 7.12-7.15 (m, 2H), 2.36 (s, 3H, CH₃), 1.32 (br s, 12H, 4 CH₃ of BPin), 1.27 (br s, 12H, 4 CH₃ of BPin); ¹³C NMR {¹H} (CDCl₃, 125 MHz): δ 152.2 (C), 139.6 (CH), 136.7 (C), 133.8 (C), 128.9 (2 CH), 128.4 (2 CH), 84.1 (2 C), 83.9 (2 C), 24.7 (4 CH₃ of BPin), 24.5 (4 CH₃ of BPin), 21.2 (CH₃); ¹¹B NMR (CDCl₃, 96 MHz): δ 29.8; FT-IR (neat) \tilde{ν}ₘₐₓ: 2978, 2932, 1537, 1473, 1373, 1331, 1309, 1261, 1140, 1037, 958, 854, 819, 669 cm⁻¹; GC-MS (EI) m/z (% relative intensity): M⁺ 426 (100), 340 (9), 310 (9); Anal. Calcd for C₂₃H₃₂B₂O₄S: C, 64.82; H, 7.57. Found: C, X; H, Y.

4. Bromination:

### a. Bromination of 2-5-Dimethyl-3-(4,4,5,5-tetramethyl-1,3, 2-dioxaborolyl)-thiophene

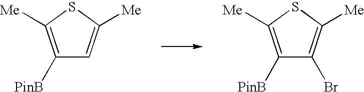

2-5-Dimethyl-3-(4,4,5,5-tetramethyl-1,3,2-dioxaborolyl)-thiophene (238 mg, 1 mmol, 1 equiv) was dissolved in 2 mL of CHCl₃ in a 20 mL scintillation vial equipped with a magnetic stirring bar. Bromine (160 mg, 1 mmol, 1 equiv, dissolved in 2 mL of CHCl₃) was added drop-wise during two minutes. The reaction was then quenched with water. The product was extracted in CH₂Cl₂ (3×20 mL) and dried over MgSO₄. Column chromatography (hexane/CH₂Cl₂ 1:1, R_{f}=0.7) furnished the desired product as a white solid (260 mg, 82%, mp 55-56° C.). ¹H NMR (CDCl₃, 300 MHz): δ 2.54 (s, 3H, CH₃), 2.29 (s, 3H, CH₃), 1.32 (br s, 12H, 4 CH₃ of BPin); ¹³C NMR {¹H} (CDCl₃, 125 MHz): δ 147.9 (C), 131.2 (C), 113.1 (C), 83.5 (2 C), 24.8 (4 CH₃ of BPin), 16.2 (CH₃), 14.5 (CH₃); ¹¹B NMR (CDCl₃, 96 MHz): δ 29.5; FT-IR (neat) \tilde{ν}ₘₐₓ: 2978, 2922, 1537, 1377, 1315, 1234, 1143, 852 cm⁻¹; GC-MS (EI) m/z (% relative intensity): M⁺ 317 (46), 318 (84), 316 (81), 303 (11) 301 (10), 261 (100), 259 (99), 237 927), 195 (38), 180 (41); Anal. Calcd for C₁₂H₁₈BBrO₂S: C, 45.46; H, 5.72. Found: C, 45.54; H, 5.91.

General Procedure E (Substitution of TMS with Br):

TMS group was replaced with Bromine by employing the literature conditions used for aromatic bromination.³ Substrate (1 mmol, 1 equiv) was added to a 20 mL scintillation vial equipped with a magnetic stirring bar. N-bromosuccinamide (1 mmol, 1 equiv) was added in to the vial. Acetonitrile (3-5 mL) was also added to the vial. The reaction mixture was stirred at room temperature and was monitored by GC-FID/MS. After the completion of the reaction, the volatile materials were removed on a rotary evaporator and the crude product was passed through a short silica plug to afford the brominated product.

### b. Bromination of 2-Chloro-3-(4,4,5,5-tetramethyl-1,3,2-dioxaborolyl)-5-trimethylsilyl-thiophene

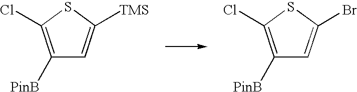

The general procedure E was applied to 2-Chloro-3-(4,4,5,5-tetramethyl-1,3,2-dioxaborolyl)-5-trimethylsilyl-thiophene (317 mg, 1 mmol) for 12 h. The product was isolated as a white solid (295 mg, 91%, mp 51-53° C.). ¹H NMR (CDCl₃, 300 MHz): δ 7.10 (s, 1H), 1.30 (br s, 12H, CH₃ of BPin); ¹³C NMR I{H} (CDCl₃, 125 MHz): δ 139.6 (C), 134.9 (CH), 108.3 (C), 84.1 (2 C), 24.8 (4 CH₃ of BPin); ¹¹B NMR (CDCl₃, 96 MHz): δ 28.5; FT-IR (neat) \tilde{ν}ₘₐₓ: 2978, 1530, 1427, 1373, 1311, 1253, 1142, 1028, 962, 848, 883, 848, 692 cm⁻¹; GC-MS (EI) m/z (% relative intensity): M⁺ 323 (48), 324 (100), 322 (81), 309 (21), 307 (14), 289 (38), 287 (36) 208 (23), 166 (22); Anal. Calcd for C₁₀H₁₃BBrClO₂S: C, 37.13; H, 4.05; Found: C, 37.25; H, 4.19.

### c. Bromination of 2-Chloro-3-(3′-methylphenyl)-5-trimethylsilyl-thiophene

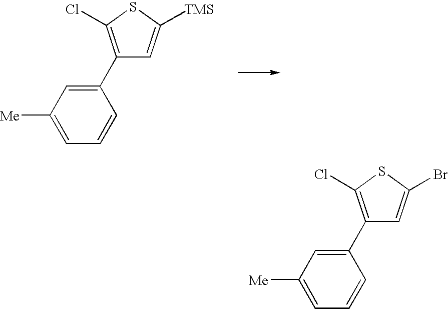

The general procedure E was applied to 2-chloro-3-(3′-methylphenyl)-5-trimethylsilyl-thiophene (280 mg, 1 mmol) for 12 h. The product was isolated as a colorless liquid (261 mg, 91%). ¹H NMR (CDCl₃, 300 MHz): δ 7.29-7.31 (m, 3H), 7.15-7.18 (m, 1H), 7.02 (s, 1H), 2.38 (s, 3H, CH₃); ¹³C NMR {¹H} (CDCl₃, 75 MHz): δ 139.3 (C), 138.2 (C), 133.1 (C), 131.2 (CH), 129.1 (CH), 128.8 (CH), 128.4 (CH), 125.5 (CH), 124.0 (C), 108.3 (C), 21.4 (CH₃); FT-IR (neat) \tilde{ν}ₘₐₓ: 3042, 2920, 2858, 1604, 1487, 1028, 972, 831, 789, 779, 700 cm⁻¹; GC-MS (EI) m/z (% relative intensity): M⁺ 287 (63), 288 (100), 290 (29), 287 (63), 251 (5), 171 (19); Anal. Calcd for C₁₁H₉BrClS: C, 45.94; H, 2.80. Found: C, 45.96; H, 2.79.

5. Suzuki Coupling of Heteroarylboronate esters:

a. One Pot Borylation/Suzuki coupling of 2-methylthiophene.

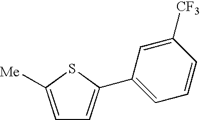

The general procedure A was applied to 2-methylthiophene (484 μL, 491 mg, 5 mmol, 1 equiv) and HBPin (870 μL, 768 mg, 6.00 mmol, 1.20 equiv) in a Schlenk flask for 0.5 h. The reaction mixture was pumped down under high vacuum for 0.5 h to remove the volatile materials. Pd(PPh₃)₄ (116 mg, 0.10 mmol, 2 mol %), 3-bromo-benzotrifluoride (837 μL, 1350 mg, 6.00 mmol, 1.2 equiv), and DME (6 mL) were added to the Schlenk flask inside the glove box. The Schlenk flask was then brought out of the glove box and attached to a Schlenk line. K₃PO₄.nH₂O (1592 mg, 1.50 equiv) was added under N₂ counter flow to the Schlenk flask. The flask was stoppered and the mixture was heated at 80° C. for 8 h. The flask was cooled down to room temperature and 20 mL of water were added to the reaction mixture. The reaction mixture was extracted with ether (20 mL×3). The combined ether extractions were washed with brine (20 mL), followed by water (10 mL), dried over MgSO₄ before being concentrated under reduced pressure on a rotary evaporator. Column chromatography (hexanes, R_{f} 0.5) furnished the product as white semi solid (1026 mg, 85% yield). ¹H NMR (CDCl₃, 500 MHz): δ 7.77 (t, J=0.8 Hz, 1H), 7.68 (d, J=7.6 Hz, 1H), 7.42-7.48 (m, 2H), 7.15 (d, J=3.5 Hz, 1H), 6.73-6.75 (m, 1H), 2.51 (s, 3H, CH₃); ¹³C NMR {¹H} (CDCl₃, 125 MHz): δ 140.7 (C), 140.1 (C), 135.5 (C), 131.2 (q, ²J_{C-F}=32.6 Hz, C), 129.3 (CH), 128.5 (CH), 126.4 (CH), 124.1 (q, ¹J_{C-F}=273 Hz, CF₃), 124.0 (CH), 123.4 (q, ³J_{C-F}=3.6 Hz, CH), 122.0 (q, ³J_{C-F}=3.6 Hz, CH), 15.4 (CH₃); FT-IR (neat) \tilde{ν}ₘₐₓ: 3073, 2922, 2865, 1497, 1340, 1325, 1165, 1126, 1074, 790, 694 cm⁻¹; GC-MS (EI) m/z (% relative intensity): M⁺ 242 (100), 223 (4), 173 (6); Anal. Calcd for C₁₂H₉F₃S: C, 59.49; H, 3.74. Found: C, 59.38; H, 3.56.
b. One Pot Borylation/Suzuki coupling of 2-chloro-5-Trimethylsilylthiophene.

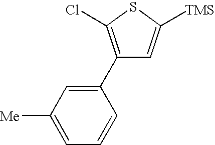

The general procedure A was applied to 2-chloro-5-trimethylsilylthiophene (382 mg, 2 mmol, 1 equiv) and HBPin (435 μL, 384 mg, 3.00 mmol, 1.50 equiv) in a Schlenk flask for 10 h. The reaction mixture was pumped down under high vacuum for 1 h to remove the volatile materials. Pd(PPh₃)₄ (46 mg, 2 mol %), 3-bromo-toluene (291 μL, 410 mg, 2.40 mmol, 1.2 equiv), and DME (3 mL) were added to the Schlenk flask inside the glove box. The Schlenk flask was then brought out of the glove box and attached to a Schlenk line. K₃PO₄.nH₂O (637 mg, 1.50 equiv) was added under N₂ counter flow to the Schlenk flask. The flask was stoppered and the mixture was heated at 80° C. for 6 h. The flask was cooled down to room temperature and 5 mL of water were added to the reaction mixture. The reaction mixture was extracted with ether (10 mL×3). The combined ether extractions were washed with brine (10 mL), followed by water (10 mL), dried over MgSO₄ before being concentrated under reduced pressure on a rotary evaporator. Column chromatography (hexanes, R_{f} 0.5) furnished the product as a colorless liquid (369 mg, 66% yield). ¹H NMR (CDCl₃, 300 MHz): δ 7.27-7.37 (m, 3H), 7.13-7.16 (m, 1H), 7.12 (s, 1H), 2.39 (s, 3H, CH₃), 0.31 (s, 9H, 3 CH₃ of TMS); ¹³C NMR {¹H} (CDCl₃, 75 MHz): δ 139.6 (C), 138.2 (C), 138.0 (C), 135.3 (CH), 134.3 (C), 129.3 (C), 129.1 (CH), 128.29 (CH), 128.27 (CH), 125.6 (CH), 21.5 (CH₃), −0.3 (3 CH₃ of TMS); FT-IR (neat) \tilde{ν}ₘₐₓ: 3040, 2957, 2922, 1606, 1408, 1252, 993, 839, 781, 756, 700, 630 cm⁻¹; GC-MS (EI) m/z (% relative intensity): M⁺ 280 (49), 282 (19), 266 (100), 267 (48); Anal. Calcd for C₁₄H₁₇ClSSi: C, 59.86; H, 6.10. Found: C, 59.56; H, 6.21.

### REFERENCES

^{a} Perepichka, I. F.; Perepichka, D. F.; Meng, H.; Wudl, F. Adv. Mater. 2005, 17, 2281-2305.
^{b} Leclerc, M.; Ho, H. A. Synlett 2004, 380-387.
^{c} Cecchetti, V.; Tabarrini, O.; Sabatini, S. Curr. Top. Med. Chem. 2006, 6, 1049-1068.
^{d} Sperry, J. B.; Wright, D. L. Curr. Opin. Drug Discov. Dev. 2005, 8, 723-740.
^{e} Ishiyama, T.; Takagi, J.; Yonekawa, Y.; Hartwig, J. F.; Miyaura, N. Adv. Synth. Catal. 2003, 345, 1103-1106.
^{f} Takagi, J.; Sato, K.; Hartwig, J. F.; Ishiyama, T.; Miyaura, N. Tetrahedron Lett. 2002, 43, 5649-5651.
^{g} Liversedge, I. A.; Higgins, S. J.; Giles, M.; Heeney, M.; McCulloch, I. Tetrahedron Lett. 2006, 47, 5143-5146.
^{h} Chotana, G. A.; Rak, M. A.; Smith, M. R., III J. Am. Chem. Soc. 2005, 127, 10539-10544.
ⁱ Uson, R.; Oro, L. A.; Cabeza, J. A. Inorg. Synth. 1985, 23, 126-130.
^{j} Merola, J. S.; Kacmarcik, R. T. Organometallics 1989, 8, 778-784.
^{k} Barton, T. J.; Hussmann, G. P. J. Org. Chem. 1985, 50, 5881-5882.
^{l} Liska, R. Heterocycles 2001, 55, 1475-1486.
^{m} Wu, X.; Rieke, R. D. J. Org. Chem. 1995, 60, 6658-6659.
ⁿ Wu, R.; Schumm, J. S.; Pearson, D. L.; Tour, J. M. J. Org. Chem. 1996, 61, 6906-6921.
^{o} Boller, T. M.; Murphy, J. M.; Hapke, M.; Ishiyama, T.; Miyaura, N.; Hartwig, J. F. J. Am. Chem. Soc. 2005, 127, 14263-14278.

The thiophenes of the present invention are intermediates to thiophene polymers useful in electronic applications including photovoltaic, sensory, light emitters and field effect transistors.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the Claims attached herein.

We claim:

1. A process for producing a boryl-mono-, di- or tri-substituted thiophene (I), which comprises:
(a) reacting a mono-, di- or tri-substituted thiophene (II) in a reaction mixture with a non-reactive solvent selected from, but not limited to, aliphatic hydrocarbons and ethers at elevated temperatures between about 0 and 150° C. with an HB or B—B organic compound, in the presence of a catalytically effective amount of an iridium complex catalytic composition comprising an iridium complex of the formula: (BY)ₙ—Ir-(ligand)ₘ where n is equal to one to five and m is equal to one to three, excluding hydrogen, bonded to the iridium BY is a boron moiety and the ligand is selected from the group consisting of a phosphorus organic ligand, an organic amine, an imine, a nitrogen heterocycle, and an ether wherein the ligand is at least in part bonded to the iridium, to form the mono-, di- or tri-substituted thiophene (I) in the reaction mixture; and
(b) evaporating the solvent and portions of the reaction mixture which are volatile from the reaction mixture to produce the boryl-mono-, di- or tri-substituted thiophene (I).

2. A process for producing a 2-boryl-5-substituted thiophene (I), which comprises:
(a) reacting a thiophene (II) with an HB or B—B organic compound in a reaction mixture with a non-reactive first solvent which is a non-solvent for the 2-boryl-5-substituted thiophene (I) at elevated temperatures between about 0 and 150° C. in the presence of a catalytically effective amount of an iridium complex catalytic composition comprising an iridium complex of the formula: (BY)ₙ—Ir-(ligand)ₘ where n is equal to one to five and m is equal to one to three, excluding hydrogen, bonded to the iridium BY is a boron moiety and the ligand is selected from the group consisting of a phosphorus organic ligand, an organic amine, an imine, a nitrogen heterocycle, and an ether wherein the ligand is at least in part bonded to the iridium, in a molar ratio of complex to ligand between 1 to 3 and 1 to 1, wherein the ligand is at least in part bonded to the iridium, to form the 2-boryl-5-substituted thiophene (I);
(b) evaporating the first solvent and portions of the reaction mixture which are volatile from the reaction mixture;
(c) dissolving the 2-boryl-5-substituted thiophene in a second solvent; and
(d) isolating the 2-boryl-5-substituted thiophene (I) from the second solvent.

3. A 2-boryl-5-substituted thiophene (I) wherein there is at least one ring substituent in the 5 position other than hydrogen selected from the group consisting of boryl, halo other than fluoro, cyano, alkyl, alkoxy, thioalkyl, amino alkyl, acyl, alkyl aminoacyl, trifluoro alkyl, aryl, alkyl silyl, and containing 1 to 8 carbon atoms except for the halo group and boryl group and wherein the boryl group is derived from HBPin or B₂Pin.

4. A process for producing 2-boryl-5-substituted thiophene (I), which comprises:
(a) reacting a 2-boryl-5-substituted thiophene (II) with HBPin or B₂Pin₂, in a reaction mixture with a non-reactive first solvent which is a non-solvent for the 2-boryl-5-substituted thiophene (I) at elevated temperatures between about 0 and 150° C. in the presence of a catalytically effective amount of an iridium complex catalytic composition comprising an iridium complex of the formula: (BY)ₙ—Ir-(ligand)ₘ where n is equal to one to five and m is equal to one to three, excluding hydrogen, bonded to the iridium BY is a boron moiety and the ligand is selected from the group consisting of a phosphorus organic ligand, an organic amine, an imine, a nitrogen heterocycle, and an ether wherein the ligand is at least in part bonded to the iridium, in a molar ratio of complex to ligand between 1 to 3 and 1 to 1, and wherein the ligand is at least in part bonded to the iridium, to form the 2-boryl-5-substituted thiophene (I) in the reaction mixture; and
(b) evaporating the solvent and portions of the reaction mixture which are volatile from the reaction mixture to produce the 2-boryl-5-substituted thiophene (I).

5. The process of claim 4 wherein there is at least one ring substituent for 5-substituted other than hydrogen selected from the group consisting of boryl, halo other than fluoro, cyano, alkyl, alkoxy, thioalkyl, amino alkyl, acyl, alkyl aminoacyl, trifluoro alkyl, aryl, alkyl silyl, and containing 1 to 8 carbon atoms except for the halo group and boryl group and the boryl group is derived from HBPin or B₂Pin.

6. The process of claims 4 or 5 wherein the ligand is selected from the group consisting of: 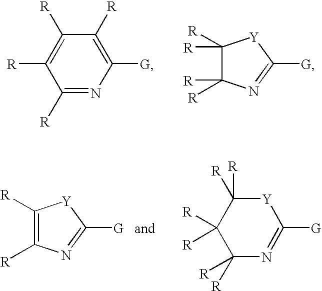
wherein R are each selected from the group consisting of hydrogen, linear alkyl containing 1 to 8 carbon atoms, branched alkyl containing 1 to 8 carbons, and a carbon in a cyclic structure, Y is a carbon, oxygen, nitrogen, or sulfur containing moiety, and G is a heteroatom containing group, multiple atom chain, or multiple atom ring.

7. The process of claims 4 or 5 wherein the ligand is selected from the group consisting of: 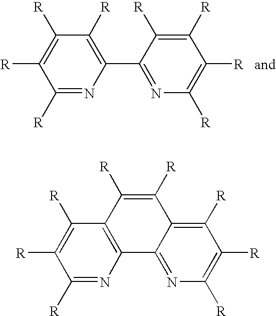
wherein R are each selected from the group consisting of hydrogen, linear alkyl containing 1 to 8 carbon atoms, branched alkyl containing 1 to 8 carbons, and a carbon in a cyclic structure.

8. The process of claims 4 or 5 wherein the ligand is selected from the group consisting of: 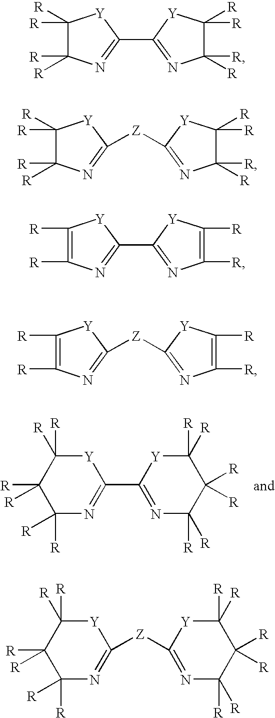
wherein R are each selected from the group consisting of hydrogen, linear alkyl containing 1 to 8 carbon atoms, branched alkyl containing 1 to 8 carbons, and a carbon in a cyclic structure, Y is a carbon, oxygen, nitrogen, or sulfur containing moiety, and Z is a carbon, oxygen, nitrogen, sulfur, or boron containing moiety or a multiple atom chain containing a carbon, oxygen, nitrogen, sulfur, or boron containing moiety.

9. The process of claims 1 or 2 wherein the HB or B—B organic compound is HBPin or B₂Pin₂.

10. The process of claims 1 or 2 wherein the complex is an iridium complex of [Ir(OMe)ₙ(CD)]₂, where COD is 1,5-cyclooctadine complexed with 4,4-di-t-butyl-2,2′bipyridine (d^{t}bpy).

11. The process of any one of claims 1, 2 or 4 wherein the ligand is bisoxazoline.
